(19) United States
(12) Patent Application Publication
(10) Pub. No.: US 2024/0358594 A1
(43) Pub. Date: Oct. 31, 2024

(54) DEVICES, SYSTEMS, AND METHODS FOR COMBINING AND/OR DELIVERING INJECTABLE MATERIALS

(71) Applicants: BOSTON SCIENTIFIC SCIMED, INC., MAPLE GROVE, MN (US); BOSTON SCIENTIFIC MEDICAL DEVICE LIMITED, Galway (IE)

(72) Inventors: Nitesh Ghananil Baviskar, Kalyan West (IN); Sanjay Kumar Chandra, Pune (IN); Benjamin Cleveland, Bellingham, MA (US); Katie Knowles, Providence, RI (US); Marshall O'Hearn, Davenport, FL (US)

(73) Assignees: BOSTON SCIENTIFIC SCIMED, INC., MAPLE GROVE, MN (US); BOSTON SCIENTIFIC MEDICAL DEVICE LIMITED, Galway (IE)

(21) Appl. No.: 18/646,296

(22) Filed: Apr. 25, 2024

Related U.S. Application Data
(60) Provisional application No. 63/462,116, filed on Apr. 26, 2023.

(51) Int. Cl. A61J 1/20; A61M 5/19; A61N 5/10

(52) U.S. Cl. CPC A61J 1/2096; A61M 5/19; A61M 2207/00; A61N 5/10

(57) ABSTRACT

Devices, systems, and methods for combining and/or delivering an injectable material to a patient. A first device having a first nozzle is moved rotationally and/or generally axially with respect to a second device having a first nozzle port to seat the first nozzle with respect to the first nozzle port. The second device has a nozzle pocket positioned, sized, shaped, configured, and/or dimensioned with respect to the first nozzle port to guide axial and/or rotational relative movements of the first injectable material transfer device and the second injectable material transfer device to guide the first nozzle into alignment with the first nozzle port, such as to be seated with respect to the first nozzle port.

### CROSS-REFERENCE TO RELATED APPLICATIONS

This application claims the benefit of priority under 35 U.S.C. § 119 to U.S. Provisional Application No. 63/462,116, filed Apr. 26, 2023, the entire disclosure of which is hereby incorporated by reference herein for all purposes.

### FIELD

The present disclosure relates generally to the field of devices for delivering materials, such as injectable materials, to a patient, and associated systems and methods. More particularly, the present disclosure relates to devices for combining components of injectable materials, and associated systems and methods.

### BACKGROUND

Various forms of cancer and other medical conditions are treated by local application of radiation therapy. However, various risks may accompany radiation therapy. Since the conception of conformal radiotherapy, physicians have paid attention to the radiation dose delivered to the target and surrounding tissues. Investigators have been able to correlate side effects to the amount of tissue receiving a certain radiation dose. And yet, time, distance, and shielding affect the dose that is delivered. The less time an area is exposed to radiation, the less dose is delivered. The greater the distance from the radiation, the less dose is delivered. Filler materials may be injected into a treatment area to provide a shield to tissue surrounding the target of the radiation therapy. For instance, numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Various systems provide filler material to treatment sites to decrease the radiation dose to tissue surrounding radiation target sites (e.g., to shield the rectum during radiotherapy for prostate cancer). Such filler materials are often reactive, and therefore are generally combined/mixed immediately prior to or even during delivery to the patient. Various systems are known for combining/mixing (e.g., in vitro) filler materials injected into radiation treatment areas. However, most such systems include numerous subcomponents, are complex to assemble, and are susceptible to filler mixing errors prior to delivery within a patient at a treatment site. Various challenges posed by such mixing systems may result in errors and mishaps which lead unnecessarily to increased procedure time, and increased procedure costs. Solutions to these and other issues presented by combining and delivering injectable materials would be welcome in the art.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an injectable material delivery system is configured to establish fluid communication between first and second injectable material transfer devices thereof. In some aspects, the system includes a first injectable material transfer device having a first nozzle, and a second injectable material transfer device having a first nozzle port configured to be seated with respect to the first nozzle of the first injectable material transfer device, and a first nozzle pocket configured to guide the first nozzle of the first injectable material transfer device as the first injectable material transfer device and the second injectable material transfer device are brought together generally axially and are rotated with respect to each other to seat the first nozzle of the first injectable material transfer device with respect to the first nozzle port of the second injectable material transfer.

In some aspects, the first injectable material transfer device and the second injectable material transfer device have engaging elements configured to couple the first injectable material transfer device and the second injectable material transfer device by relative axial and/or rotational movements of the first injectable material transfer device and the second injectable material transfer device, with the guidance of the first nozzle pocket. In some aspects, the system includes a biasing element positioned between the first injectable material transfer device and the second injectable material transfer device to bias the first injectable material transfer device and the second injectable material transfer device with respect to each other to lock the engaging elements of the first injectable material transfer device and the second injectable material transfer device with respect to each other. In some aspects, the system includes a third injectable material transfer device having engaging elements configured to be similar to engaging elements of the second injectable material transfer device to engage engaging elements of the first injectable material transfer device. In some aspects, the third injectable material transfer device has a first nozzle port configured to be seated with respect to the first nozzle of the first injectable material transfer device, and a first nozzle pocket configured to guide the first nozzle of the first injectable material transfer device as the first injectable material transfer device and the third injectable material transfer device are brought together generally axially and are rotated with respect to each other to seat the first nozzle of the first injectable material transfer device with respect to the first nozzle port of the third injectable material transfer device. In some aspects, the system includes a fourth injectable material transfer device having engaging elements configured to be similar to engaging elements of the first injectable material transfer device to engage engaging elements of the second injectable material transfer device.

The first injectable material transfer device may be a device defining a material chamber therein and having at least one nozzle through which material is ejected therefrom or aspirated therein; a multi-chamber device; a single-chamber device; a device defining a nozzle port with respect to which a nozzle of another device may be seated in fluid communication therewith; a vial adaptor; an injection system; a pretreatment system; or an adaptor device or system coupling or facilitating coupling of a first device with at least one nozzle with a second device with at least one nozzle port.

In some aspects, the first injectable material transfer device is a multi-chamber device defining a first chamber and a second chamber therein; the first injectable material transfer device has a second nozzle; the first nozzle of the first injectable material transfer device is in fluid communication with the first chamber of the first injectable material transfer device; and the second nozzle of the first injectable material transfer device is in fluid communication with the second chamber of the first injectable material transfer device. In some aspects, the first nozzle port of the second injectable material transfer device is configured to be seated with respect to the first nozzle of the first injectable material transfer device; the second injectable material transfer device has a second nozzle port configured to be seated with respect to the second nozzle of the first injectable material transfer device; and the second injectable material transfer device has a second nozzle pocket configured to guide the second nozzle of the first injectable material transfer device to seat with respect to the second nozzle port of the second injectable material transfer device as the first injectable material transfer device and the second injectable material transfer device are brought together generally axially and are rotated with respect to each other to seat the second nozzle of the first injectable material transfer device with respect to the second nozzle port of the second injectable material transfer.

In some aspects, the second injectable material transfer device comprises an injection system comprising a material delivery device configured to deliver injectable material ejected from the first injectable material transfer device and through the second injectable material transfer device to a patient.

In some aspects, the first injectable material transfer device has a barrel defining a first chamber therein, and a plunger assembly movable with respect to the first chamber to eject material from or to aspirate material into the first chamber.

In some aspects, the system includes a vial adaptor having a vial-receiving chamber configured to receive a vial therein; a first nozzle port configured to be seated with respect to the first nozzle of the first injectable material transfer device; and a first nozzle pocket configured to guide the first nozzle of the first injectable material transfer device as the first injectable material transfer device and the vial adaptor are brought together generally axially and are rotated with respect to each other to seat the first nozzle of the first injectable material transfer device with respect to the first nozzle port of the vial adaptor.

In some aspects, the system includes an adaptor system having a single nozzle port configured to be fluidly coupled with a single-nozzle device, and a first nozzle configured to be guided by the first nozzle pocket of the injection system as the adaptor system and the injection system are brought together generally axially and are rotated with respect to each other to seat the first nozzle of the adaptor system with respect to the first nozzle port of the injection system.

In accordance with various principles of the present disclosure, an injectable material transfer device is configured for coupling with another injectable material transfer device having a first nozzle. In some aspects, the injectable material transfer device includes a first nozzle port configured to be seated with respect to a first nozzle of the other injectable material transfer device; and a first nozzle pocket configured to guide the first nozzle of the other injectable material transfer device as the injectable material transfer device and the other injectable material transfer device are brought together generally axially and are rotated with respect to each other to seat the first nozzle of the other injectable material transfer device with respect to the first nozzle port of the injectable material transfer device.

In some aspects, the device further includes engaging elements configured to engage the device with engaging elements of the other injectable material transfer device, wherein the device and the other injectable material transfer device move generally axially as well as rotationally with respect to each other as the engaging elements of the device and the engaging elements of the other injectable material transfer device are moved with respect to each other.

In accordance with various principles of the present disclosure, a method, for coupling a first injectable material transfer device with a second injectable material transfer device, includes advancing a nozzle of the first injectable material transfer device axially into a nozzle pocket of the second injectable material transfer device; and moving the first injectable material device and the second injectable material transfer device rotationally and generally axially within nozzle pockets configured to guide movement of the first nozzle into alignment and to seat with a first nozzle port defined in the second injectable material transfer device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to facilitate injection into a patient. For purposes of clarity and simplicity, not every element is labeled in every figure, nor is every element of each embodiment shown where illustration is not necessary to allow those of ordinary skill in the art to understand the disclosure. Moreover, reference characters may indicate elements in some figures which are illustrated in other figures, and which, for the sake of brevity, are described only with reference to the other figures. It will be appreciated that common features in the drawings are identified by common reference characters and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Finally, certain features in one embodiment may be used across different embodiments and are not necessarily individually labeled when appearing in different embodiments.

The detailed description will be better understood in conjunction with the accompanying drawings, wherein like reference characters represent like elements, as follows:

FIG. 1 illustrates a perspective view of an example of an embodiment of a system for combining and/or delivering injectable materials to a patient formed in accordance with various principles of the present disclosure.

FIG. 2 illustrates an exploded perspective view of an example of an embodiment of a multi-chamber system formed in accordance with various principles of the present disclosure.

FIGS. 3A-3I illustrate various stages of preparing an injectable material, in accordance with various principles of the present disclosure, with an example of an embodiment of a multi-chamber system such as illustrated in FIG. 2.

FIG. 4 illustrates a perspective view of a vial adapter positioned for coupling with a barrel of a multi-chamber device such as illustrated in FIG. 3A.

FIG. 5 illustrates a proximal end view of the vial adaptor of FIG. 4, with the nozzles of the multi-chamber device illustrated in phantom as positioned, in FIG. 4, with respect to the vial adaptor.

FIGS. 6A, FIG. 6B, and FIG. 6C illustrate perspective views of various stages of relative movements bringing the vial adapter and the multi-chamber device illustrated in FIG. 3A into the position illustrated in FIG. 3B.

FIG. 6Ai illustrates a cross-sectional view along line VIAi-VIAi of FIG. 6A.

FIG. 6Aii illustrates a cross-sectional view along line VIAii-VIAii of FIG. Ai.

FIG. 6Bi illustrates a cross-sectional view along line VIBi-VIBi of FIG. 6B.

FIG. 6Bii illustrates a cross-sectional view along line VIBii-VIBii of FIG. 6Bi.

FIG. 6Ci illustrates a cross-sectional view along line VICi-VICi of FIG. 6C.

FIG. 6Cii illustrates a cross-sectional view along line VICii-VICii of FIG. 6Ci.

FIG. 7 illustrates a proximal perspective view of a vial adaptor such as illustrated in FIG. 4, but taken from a perspective angle closer to the proximal end than as illustrated in FIG. 4.

FIG. 8A illustrates a perspective view of an example of an embodiment of a multi-chamber device positioned for engagement with an example of an embodiment of an injection system, each formed in accordance with various principles of the present disclosure.

FIG. 8B illustrates the multi-chamber device and injection system illustrated in FIG. 8A fluidly coupled for delivery of injectable material, from within the multi-chamber device and via the injection system, to a patient.

FIG. 8C illustrates the plunger assembly of the multi-chamber device illustrated in FIG. 8B advanced to deliver injectable material out from the multi-chamber device and through the injection system for delivery to a patient.

FIG. 9 illustrates a perspective view of the injection system illustrated in FIG. 8A but taken from a perspective angle closer to the proximal end than as illustrated in FIG. 8A.

FIG. 10 illustrates a proximal end view of the injection system of FIG. 8A, with the nozzles of the multi-chamber device illustrated in phantom as positioned, in FIG. 8A, with respect to the injection system.

FIG. 11 illustrates a distal perspective view of an example of an embodiment of a support portion of an injection system such as illustrated in FIG. 9.

FIG. 12 illustrates an exploded perspective view of an example of an embodiment of an injection system and an example of an embodiment of an adaptor system, each formed in accordance with various principles of the present disclosure for engagement with the other.

FIG. 13A illustrates an injectable material transfer device positioned to be fluidly coupled with an injection system such as illustrated in FIG. 1 and FIG. 12, with the assistance of an adaptor system such as illustrated in FIG. 1 and in FIG. 12.

FIG. 13B illustrates the injectable material transfer device illustrated in FIG. 13A fluidly coupled with the injection system and adaptor system illustrated in FIG. 13A.

FIG. 13C illustrates the injectable material transfer device illustrated in FIG. 13B in a position for ejecting material therefrom and through the injection system and adaptor system illustrated in FIG. 13B.

FIG. 13D illustrates the injectable material transfer device illustrated in FIG. 13C being detached from the injection system and adaptor system illustrated in FIG. 13C after ejection of material therefrom.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical procedures involve delivery (e.g., injection) of injectable material(s) into the body before, during, or after the procedure. Preferably, the injectable material is biocompatible, and optionally biodegradable. The injectable material may serve a variety of purposes, including, without limitation, differentiating tissue (e.g., by creating a “bleb” or other raised or swelled region to distinguish an anatomical region), spacing anatomical structures from one another, otherwise affecting (e.g., shielding, coating, covering, modifying, etc.) an anatomical structure, etc. It will be appreciated that the term “tissue” is a broad term that encompasses a portion of or site within a body: for example, a group of cells, a group of cells and interstitial matter, an organ, a portion of an organ, an anatomical portion of a body, e.g., a rectum, ovary, prostate, nerve, cartilage, bone, brain, or portion thereof, etc. Moreover, reference may be made, herein, to a “target” in referring to an area of the patient's body at which a procedure is to be performed. However, it will be appreciated that such reference is to be broadly understood and is not intended to be limited to tissue or to particular procedures. Finally, reference may be made to target tissue, target location, target site, target tissue site, anatomical site, delivery site, deployment site, injection site, treatment site, etc., including combinations thereof and other grammatical forms thereof, interchangeably and without intent to limit.

Certain specific aspects of the present disclosure relate to placing an injectable material between target tissue to be treated and other tissues. For the sake of convenience, and without intent to limit, reference is made to an injectable material, such as a filler, including, without limitation, a gel composition. The injectable material may be delivered within the patient to displace the tissue relative to a tissue that is to be treated by a therapeutic procedure or otherwise (e.g., not necessarily therapeutic). Certain aspects of the present disclosure include displacing and/or shielding a tissue to protect the tissue against possible side effects of treatment of a target tissue, such as the effects of a treatment involving radiation or cryotherapy. In some aspects, the injectable material may displace anatomical tissue and/or may increase the distance between the target tissue and other tissues. For instance, if the target tissue is to be irradiated, the injectable material may space other tissues from the target tissue so that the other tissues are exposed to less radiation and/or are shielded from the radiation. In some aspects, the injectable material is injected as a filler in a space between tissues. A first tissue may then be treated by radiation, while the injectable material reduces passage of radiation therethrough into a second tissue. The first tissue may be irradiated while the second tissue, spaced by the injectable material, receives less radiation than it would have in the absence of the injectable material. An effective amount of an injectable material may be injected into a space between a first tissue to be treated and a second tissue which can be a critically sensitive organ. For instance, in the context of treatment of prostate cancer, an injectable material may be injected into the Denonvilliers' space (a region between the rectum and prostate) to create additional space between the rectum and prostate and/or to shield the rectum during treatment, thereby reducing rectal radiation dose and associated side effects.

In some aspects of the present disclosure, components of an injectable material are combined by a system formed in accordance with various principles of the present disclosure, and injected into or near a target site. It will be appreciated that terms such as combine, mix, blend, etc., (including other grammatical forms thereof) may be used interchangeably herein without intent to limit unless otherwise indicated. Reference is accordingly made herein to a combining system generically, without intent to specifically require active combining/mixing.

In accordance with various principles of the present disclosure, the injectable material may be a filler such as a hydrophilic polymer, a gel, a hydrogel, etc. For instance, the injectable material can include polymeric materials which are capable of forming a hydrogel upon crosslinking. Optionally, the polymer forms a hydrogel within the body. A hydrogel is defined as a substance formed when a polymer (natural or synthetic) is crosslinked via covalent, ionic, or hydrogen bonds to create a three-dimensional structure which entraps water molecules to form a gel. Naturally-occurring and synthetic hydrogel-forming polymers, polymer mixtures, and copolymers may be utilized as hydrogel precursors. In some aspects, the hydrogel can be formed by a composition formed by two or more constituents/components (e.g., mixing accelerant fluid, diluent, and polyethylene glycol (PEG) together), and may include one or more polysaccharide compounds or a salt thereof. For example, the composition may include a cellulose compound such as carboxymethyl cellulose (CMC) or salt thereof (e.g., CMC sodium), xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be crosslinked with a suitable crosslinking compound, such as butanediol diglycidyl ether (BDDE). In some aspects, the polysaccharide may be a homopolysaccharide or a heteropolysaccharide.

In some aspects of the present disclosure, two or more components of an injectable material are provided separately, and are combined by a device, system, and method in accordance with various principles of the present disclosure to form a compound material to be injected into or near a target site by devices, systems, and methods in accordance with various principles of the present disclosure. The injectable material may be delivered within the patient to displace the tissue relative to a tissue that is to be treated by a therapeutic procedure or otherwise (e.g., not necessarily therapeutic). A composition to be injected into a patient may be a combination of two or more components combined by a device, system, or method formed in accordance with various principles of the present disclosure. For instance, devices, systems, or methods of the present disclosure may be used to combine a first component and a second component for injection into a patient. The first component may be a precursor, e.g., a first component to be combined with a second component to form the injectable compound. The second component may be an accelerator, an accelerant, an activating agent, a cross-linking inducing agent, a catalyst, an initiator, etc., which, upon combination with the precursor, produces the injectable compound, such as by altering the chemical composition or structure of the first component. The components may be combined prior to (e.g., immediately prior to or during) delivery (e.g., during injection) to the patient so that the injectable material does not have time to form into a structure which may be difficult to inject or otherwise deliver to the patient. As such, the combination of the first component and the second component may be such that the injectable compound attains its final desired properties/reaches its final form in situ.

In some embodiments, the injectable material is formed of a first component, a second component, and a third component. For instance, for various reasons it may be desirable to provide a first, precursor component in a solid form (e.g., to allow mixing at the time of delivery, and/or to be more stable for storage and/or transport). The first component is combinable with the third component, and the thus-formed combined composition (which may be referenced as the precursor) is then combinable with the second component once the medical professional is ready to deliver (e.g., inject) the injectable material to the patient. The second component may facilitate a crosslinking interaction between the first and third components, for example, by initiating or accelerating the crosslinking interaction of the first and third components. Typically, one or more of the components of the injectable materials are biocompatible polymers. In some aspects, one of the first component or third component is a reactive polymer, such as a cross-linkable and/or hydrophilic polymer component (e.g., PEG), and the other of the first component or third component is a diluent (e.g., mostly water) in which a solid or semi-solid form of the one of the first component or third component is dissolved or dispersed, and/or with which the one of the first component or third component is cross-linked (or at least cross-linkable, such as upon further combination with the second component), to form a precursor. The second component may be an accelerator, an accelerant, an activating agent, a catalyst, an initiator, etc. (such terms being used interchangeably herein without intent to limit), combinable and reactive with the precursor (formed from the first component and the third component) to form the desired injectable material. In one example of an embodiment, a first component, in the form of a cross-linking agent (specifically, trilysine, which contains multiple nucleophilic groups, specifically, amino groups), is mixed with a third component, in the form of a reactive polymer (specifically, PEG) that has been derivatized with reactive, optionally, electrophilic groups (specifically, succinimide ester groups), under acidic pH conditions where the succinimide ester groups and the amino groups do not react to any significant degree. When this mixture is combined with a second component, in the form of an accelerant (specifically, a basic buffer solution), the pH of the resulting mixture becomes basic, at which point the amino groups of the trilysine react with the succinimide ester groups of the PEG to form covalent bonds, thereby crosslinking the PEG and forming a hydrogel.

It will be appreciated that reference to “first”, “second”, or “third” is not intended to connote a particular nature of the material or the order in which the material is combined. As such, “first”, “second”, and “third” may be used to reference any of three components forming an injectable material in accordance with various principles of the present disclosure. A non-limiting example of such components combinable by devices, systems, or methods in accordance with various principles of the present disclosure includes a first component, such as a reactive component, a solute, etc.; a second component, such as a diluent with which the reactive component is to be combined to form a precursor; and a third component such as an accelerator combinable with the precursor to form an injectable material. The injectable material is a biocompatible material, such as a polymeric material, such as a filler, or such as a hydrogel.

In some examples, the composition may be or include a gel with a desired gel strength and/or viscosity, such as a biocompatible gel suitable for injection (e.g., through a needle), as discussed in further detail below. In one example of an embodiment, one of the components is a biocompatible polymeric component. More particularly, in one example of an embodiment, one the components is a hydrophilic polymer, which may be natural or synthetic in origin, and may be anionic, cationic, zwitterionic, or neutrally charged. Non-limiting examples of hydrophilic polymers include natural hydrophilic polymers including proteins such as collagen and polysaccharides such as gellan gum, xanthan gum, gum arabic, guar gum, locust bean gum, alginate, and carrageenans, and synthetic hydrophilic polymers such as polyethylene glycols (PEG), PEG-methacrylates, PEG-methylmethacrylates, polyvinyl alcohols, polyacrylates and polymethacrylates, polyacrylic acids and their salts, polymethacrylic acids and their salts, polymethylmethacrylates, carboxymethylcelluloses, hydroxyethylcelluloses, polyvinylpyrrolidones, polyacrylamides such as N,N-methylene-bis-acrylamides or tris(hydroxymethyl)methacrylamides. The hydrophilic polymer may be modified to provide functional groups that are reactive with functional groups of a suitable cross-linking agent, which may be a covalent or ionic cross-linking agent.

The concentrations of gelling agent(s) in a composition formed in accordance with various principles of the present disclosure maybe at least about 0.01% by weight with respect to the total weight of the composition, and at most about 2.0% by weight with respect to the total weight of the composition, including increments of about 0.01% therebetween. For instance, the concentration of gelling agent(s) may range from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, a composition formed in accordance with various principles of the present disclosure may have a viscosity of at least about 0.001 pascal-second (Pa·s), and at most about 0.100 Pa·s at a shear rate of 130 s⁻¹. For instance, the composition may have a viscosity ranging from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, a composition formed in accordance with various principles of the present disclosure may have a viscosity of at least about 0.001 Pa·s, and at most about 0.050 Pa·s at a shear rate of 768 s⁻¹. For instance, the composition may have a viscosity ranging from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The various components forming the injectable material may be kept separate until the time of the procedure in which the final composition is used for various reasons, such as to maintain stability of the individual components. In some embodiments, a multi-chamber system includes separate chambers for components to be combined to form the injectable material to be delivered to the patient by an injection system. In some embodiments, a first component and a second component are separately contained within a first chamber and a second chamber, respectively, of a multi-chamber device. A third component may be contained in a separate device defining a third chamber of the multi-chamber system. To deliver the injectable material, the components of the first and third chambers are combined within the first chamber (e.g., to form a precursor), and then the components of the first and second chambers and injected together into the patient. The multi-chamber device may or may not mix the contents of the first chamber with the contents of the second chamber. For instance, the multi-chamber device may deliver and inject the components of the injectable material to an injection system, with the injection system including a mixer component configured to mix the components from the first chamber of the multi-chamber device with the contents from the second chamber of the multi-chamber device as those contents are injected from the multi-chamber device and the injection system into the patient. The already-combined first and third components are combined with the second component to form the desired form, structure, composition, properties, etc., of the injectable material to be delivered to and/or deposited within the patient. The final form, structure, composition, properties, etc., of the injectable material may be attained once the combined components are within the patient.

The present disclosure provides devices, systems, and methods for combining components to form an injectable composition, and corresponding medical devices, systems, and methods for use thereof and/or delivery to a treatment site of a patient. According to some aspects of the present disclosure, such as described above, a multi-chamber system may include a plurality of chambers for the one or more components of the injectable material and for combinations of such components. It will be appreciated that terms such as chamber, reservoir, container, vial, lumen, etc., may be used interchangeably herein without intent to limit, to refer to elements which contain, convey, hold, transport, collect, etc., a component (fluid, particulate, liquid, solid, gas, etc.) of an injectable material. Suitable chambers may include, for example, vials, syringes (e.g., a syringe barrel compatible with a manual or automatic injection system) and other fluid containers, such as configured for use with a suitable injection system. Examples of materials suitable for chambers of devices or systems of the present disclosure include, but are not limited to, cyclic olefin polymer, polypropylene, polycarbonate, polyvinyl chloride, and glass. In some aspects, one of these materials (e.g., cyclic olefin copolymer specifically) can have a coating applied to it (such as SiO₂ coating), which is advantageous so the coating can provide a primary oxygen barrier, behave as a glass-like layer, and/or can be applied using a vapor deposition process.

A combining device or system formed in accordance with various principles of the present disclosure to combine two or more components to form an injectable material may include and/or be removably connected to one or more injection systems which are configured to deliver the injectable materials to a patient. According to some aspects of the present disclosure, the filler compositions which may be used with various systems disclosed herein, e.g., the compositions prepared by the various devices, systems, methods disclosed herein, may have sufficient strength, e.g., gel strength, to withstand the forces on the continuity of the three-dimensional configuration (e.g., gel network) of the composition, and thereby minimize the effects of such forces. In the meantime, compositions with sufficient strength to withstand forces thereon may have a viscosity suitable for injection, e.g., a viscosity that does not cause the composition to become stuck in the reservoir(s), delivery lumen, needle, or other structure in which the composition is contained or through which it passes. According to some aspects of the present disclosure, the composition may maintain its three-dimensional structure until the composition is injected into a patient (e.g., through a needle), whereupon the structure may form fragments of the original continuous, three-dimensional network. Those fragments may have a diameter corresponding to the diameter of the lumen through which it passes into the patient (e.g., the lumen of an injection needle), such that the fragments are as large as possible in-vivo to retain as much of the three-dimensional structure of the composition as possible. Injection of these larger-sized particles or fragments is believed to increase the amount of time the gel remains within the tissue.

In some examples, the injection system includes a needle. In some embodiments, the needle may be a hypodermic needle, and may range from a size of 7-gauge (4.57 mm outer diameter (OD), 3.81 mm inner diameter (ID)) to 33-gauge (0.18 mm OD, 0.08 mm ID), e.g., a size of 16-gauge (1.65 mm OD, 1.19 mm ID), 18-gauge, 21-gauge (0.82 mm OD, 0.51 mm ID), 22-gauge (0.72 mm OD, 0.41 mm ID), 23-gauge (0.64 mm OD, 0.33 mm ID), or 24-gauge (0.57 mm OD, 0.31 mm ID). According to some aspects of the present disclosure, the size of the needle may be chosen based on the viscosity and/or components of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein. Examples of materials which may be used to form the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

As noted above, compositions used with systems disclosed herein may have large particulate matter (relative to the injection system lumen) and/or a high viscosity for passage through a lumen sized to inject the material into the patient. The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lb·f to about 25 lb·f, such as from about 10 lb·f to about 20 lb·f, e.g., about 15 lb·f. The loads measured for a given gel concentration may vary for different needles and flow rates.

According to some aspects of the present disclosure, a combining device or system can be included in a kit for introducing an injectable material into a patient, whereby the injectable material can include any of a variety of suitable compositions. Kits or systems may be configured to store one or more of the components of a composition until the medical professional is ready to mix the composition for delivery to a patient. For instance, compositions, such as hydrogels, may be prepared so that the precursor(s) and any related activating agent(s) are stored in the kit with diluents as may be needed. Applicators may be used in combination with the same. Kits formed in accordance with various principles of the present disclosure can be manufactured using medically acceptable conditions and contain components that have sterility, purity, and preparation that is pharmaceutically acceptable. Solvents/solutions may be provided in the kit or separately. The kit may include one or more syringes and/or needles for mixing and/or delivery of the injectable material, and/or for additional aspects of the procedure in which the injectable material is to be used. The kit or system may comprise various components as set forth herein. For instance, a target site into which an injectable material is to be delivered may be pre-treated using one or more components of the kit. One example of a pretreatment includes hydrodissection, such as with saline, to create space for injectable material to be injected at or in the vicinity of the target tissue site. Once saline has been injected to the treatment site, a combining device or system can be connected to a needle (e.g., an 18-gauge spinal needle) to then deliver the injectable material to the treatment site. For instance, in treating prostate cancer, a 5-10 mm layer of filler (e.g., gel composition) may be injected along the posterior wall of the prostate between the prostate and rectum. Once the filler has been injected into the space between the rectum and prostate, ultrasound images can be obtained.

In accordance with various principles of the present disclosure, a combining and/or delivery system is configured to facilitate combining of components of an injectable material. In some aspects, the injectable material is a combination of a first component, a second component, and a third component, such as described above. A combining and/or delivery system formed in accordance with various principles of the present disclosure facilitates combining/mixing of the various components of an injectable material. Additionally or alternatively, a combining and/or delivery system formed in accordance with various principles of the present disclosure facilitates delivery of the injectable material to an injection system configured to deliver to and/or deposit the injectable material into (e.g., inject) a patient (e.g., to a target site within the patient). More particularly, various aspects of the present disclosure simplify assembly, alignment, mixing, dispensing, etc., of an injectable material which is combined or blended or mixed from separate components before delivery to a patient.

In some aspects, a combining and/or delivery system formed in accordance with various principles of the present disclosure includes a multi-chamber system with three distinct chambers for respective first, second, and third components of an injectable material to be delivered and injected into a patient. The present disclosure facilitates combination of the separately-provided components before a procedure, such as to form a precursor, as well as combination of the recently-combined components/precursor with another of the components of the injectable material for injection into the patient.

An example of an embodiment of a multi-chamber system disclosed herein is configured to facilitate the combining of the first, second, and third components of an injectable material before delivery to a patient. As described above, it may be desirable to provide the first, second, and third components separate from one another for combination only once a procedure is to be performed utilizing the injectable material formed by combining the first, second, and third components. The multi-chamber system may then be fluidly coupled with an injection system, formed in accordance with various principles of the present disclosure, to deliver the injectable material to a patient.

In an example of an embodiment disclosed herein, a multi-chamber system formed in accordance with various principles of the present disclosure includes a multi-chamber device having a barrel defining first and second separate chambers, and a vial defining a third chamber. The multi-chamber system further includes a vial adaptor configured to facilitate fluid coupling of the vial with the barrel to allow combining of a component within one of the two chambers of the barrel with a component within the vial. The vial adaptor and the multi-chamber device are optionally configured to facilitate secure manual coupling/engagement of the barrel of the multi-chamber device with the vial adaptor. Additionally or alternatively, the vial adaptor and the multi-chamber device are configured to facilitate manual decoupling/disengagement of the barrel of the multi-chamber device from the vial adaptor. It will be appreciated that reference herein to facilitating manual coupling, engaging, decoupling, disengaging, etc. (such terms, including other grammatical forms thereof, being used interchangeably herein without intent to limit) is to be understood as being configured to allow ready, easy, simplified movements and/or steps without the need for additional tools or complex or difficult or time-consuming steps which may otherwise interfere with expeditious use of the devices and/or systems of the present disclosure, as may be appreciated by those of ordinary skill in the art.

A combining and/or delivery system formed in accordance with various principles of the present disclosure optionally further includes an injection system configured to deliver an injectable material to a patient (e.g., to inject the injectable material into the patient). In accordance with various principles of the present disclosure, the multi-chamber device and the injection system of a combining and/or delivery system of the present disclosure optionally configured to facilitate secure fluid coupling/engagement of the multi-chamber device and the injection system so that injectable material may be ejected out from the multi-chamber device and into a patient via the injection system. In some embodiments, the injection system is configured to engage with engaging elements of the multi-chamber device which are configured to engage with engaging elements of a vial adaptor. In some embodiments, the engaging elements of the injection system are similar to or substantially the same as the engaging elements of the vial adaptor.

Various devices of a combining and/or delivery system formed in accordance with various principles of the present disclosure may be considered herein to be injectable material transfer devices. For instance, injectable material transfer devices include, without limitation, a material delivery device defining a material chamber therein and having at least one nozzle through which material is ejected therefrom or aspirated therein; a multi-chamber device; a single-chamber device; a device defining a port with respect to which a nozzle of another device may be seated to eject a material therein or therethrough; a vial; a vial adaptor; an injection system; a pretreatment system; and/or an adaptor device or system coupling or facilitating coupling of any of the aforementioned devices with another device may all be considered injectable material transfer devices. It will be appreciated that the term transfer, as used herein, should be broadly understood to encompass interactions with an injectable material, such as transferring, handling, transporting, containing, injecting, aspirating, mixing, delivering, etc., such terms (and other grammatical forms thereof) being used interchangeably herein without intent to limit, the present disclosure not being limited in this regard. In accordance with various principles of the present disclosure, a first injectable material transfer device has at least one nozzle, and a second injectable material transfer device has at least one port, the at least one nozzle being configured to seat with respect to the at least one port to fluidly couple the first and second injectable material transfer devices. In accordance with various further principles of the present disclosure, the first and second injectable material transfer devices include engaging elements configured to facilitate engaging of the first and second injectable material transfer devices to seat and fluidly communicate the at least one nozzle and the at least one nozzle port with respect to each other. In some aspects, the engaging elements of the first and second injectable material transfer devices are directly coupled with one another, without an additional coupling device or system with additional engaging elements therebetween. Finally, in accordance with even further various principles of the present disclosure, the second injectable material transfer device includes at least one nozzle pocket positioned, sized, shaped, configured, and/or dimensioned to guide the at least one nozzle into alignment with and/or to seat with respect to the at least one nozzle port. For instance, the nozzle pocket may be curved and/or may extend generally axially from the at least one port and towards the at least one nozzle to guide the at least one nozzle as the first and second injectable material transfer devices are moved rotationally and/or generally axially with respect to each other. In some aspects, the engaging elements of the first and second injectable material transfer devices are configured such that the first and second injectable material transfer devices must move rotationally and/or generally axially with respect to each other for the at least one nozzle and the at least one nozzle port to seat with respect to each other. With engaging elements configured in such manner, the at least one nozzle pocket may be positioned, sized, shaped, configured, and/or dimensioned to guide the at least one nozzle axially as well as rotationally with the relative axial and rotational movements of the first and second injectable material transfer devices in seating the at least one nozzle with respect to the at least one nozzle port. The axial extent of the at least one nozzle pocket is generally from the at least one nozzle port to the end of the second injectable material transfer device to be coupled with the first injectable material transfer device. The at least one nozzle port generally extends circumferentially with respect to a longitudinal axis LA of the second injectable material transfer device, such as for rotationally guiding the at least one nozzle as the first and second injectable material transfer devices are rotate with respect to each other.

In some aspects a multi-chamber device (e.g., a multi-chamber syringe) is configured to be fluidly coupled with one or more injectable material transfer devices, such as a vial, a vial adaptor, an injection system, etc., and the likewise the injectable material transfer device is configured to be fluidly coupled with the multi-chamber device. In some embodiments, the multi-chamber device has a barrel defining two or more chambers therein, each chamber being configured to contain an injectable material component. In accordance with various principles of the present disclosure, the multi-chamber device has a nozzle fluidly communicating with each chamber and through which the component may be ejected. Likewise, an injectable material transfer device, formed in accordance with various principles of the present disclosure for fluidly coupling with a multi-chamber device formed in accordance with various principles of the present disclosure, has a nozzle port corresponding to each nozzle of the barrel. The contents of the barrel are fluidly coupled with the injectable material transfer device via the nozzles and the nozzle ports. In accordance with various further principles of the present disclosure, the nozzles and nozzle ports are moved into fluid engagement via nozzle pockets positioned, sized, shaped, configured, and/or dimensioned with respect to the nozzle ports to guide the nozzles into alignment and/or to seat with respect to the nozzle ports.

In some aspects, at least one of the chambers of the multi-chamber device is fluidly coupled, via an associated nozzle (in fluid communication with the at least one chamber), with a corresponding nozzle port of a vial adaptor. The at least one chamber of the multi-chamber device may be fluidly coupled, via the nozzle and nozzle port, with a vial received in a vial-receiving chamber of the vial adaptor. The multi-chamber device and the vial adaptor may be coupled together by engagement of engaging elements on the multi-chamber device and the vial adaptor, such as achieved by relative generally axial and rotational movements of the multi-chamber device and the vial adaptor. In accordance with various principles of the present disclosure, the nozzle ports of the vial adaptor have nozzle pockets positioned, sized, shaped, configured, and/or dimensioned with respect to the nozzle ports to guide the nozzles of the multi-chamber device with respect to the nozzle ports of the vial adaptor, such as to be seated with respect thereto, as the multi-chamber device and vial adaptor are brought closer together axially and are rotated with respect to each other.

In some aspects, an injection system formed in accordance with various principles of the present disclosure has nozzle ports with respect to which the nozzles of a multi-chamber device are to be aligned and/or seated. Similar to the vial adaptor, the multi-chamber device and the injection system may be coupled together by engagement of engaging elements on the multi-chamber device and the injection system, such as achieved by relative generally axial and rotational movements of the multi-chamber device and the injection system. In accordance with various principles of the present disclosure, the nozzle ports of the injection system have associated nozzle pockets positioned, sized, shaped, configured, and/or dimensioned to guide the nozzles of the multi-chamber device with respect to the nozzle ports of the injection system as the multi-chamber device and injection system are brought closer together axially and are rotated with respect to each other. Once the nozzles and nozzle ports are aligned and/or seated with respect to each other, the contents of the multi-chamber device may be ejected out of the multi-chamber device and through the injection system into a patient.

A combining and/or delivery system formed in accordance with various principles of the present disclosure optionally further includes an adaptor system configured to facilitate secure coupling/engagement of another injectable material transfer device with an injection system configured for fluid coupling with two or more nozzles of a multi-chamber device. Additionally or alternatively, the adaptor system is configured to facilitate decoupling/disengagement of the other injectable material transfer device from the injection system. The injectable material transfer device may be a pretreatment system configured to deliver pretreatment materials to a patient via the injection system. In some embodiments, the injectable material transfer device has a single nozzle, and the adaptor system is configured to adapt the single nozzle for coupling with the two nozzle ports of the injection system. The adaptor system may thus have a single port for fluidly communicating with the single nozzle of the injectable material transfer device, and a nozzle associated with each nozzle port of the injection system. Nozzle pockets associated with the nozzle ports of the injection system may be positioned, sized, shaped, configured, and/or dimensioned to facilitate alignment and seating of the nozzles of the adaptor system with respect to the nozzle ports of the injection system in a similar manner as described above with respect to the alignment and seating of the multi-chamber device nozzles with respect to the injection system ports.

It will be appreciated that various concepts of the present disclosure described herein need not be limited to injectable material transfer devices with two or more nozzles and injectable material transfer devices with corresponding nozzle ports. For instance, various principles of the present disclosure may be applied to a single-nozzle-device fluidly coupled with a corresponding nozzle port formed with a nozzle pocket such as described above. Moreover, various principles of the present disclosure may be applied to devices with more than two nozzles and devices with corresponding nozzle ports. As understood herein, corresponding is intended to convey a relationship between components, parts, elements, etc., configured to interact with or to have another intended relationship with one another.

Various embodiments of combining and/or delivery devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

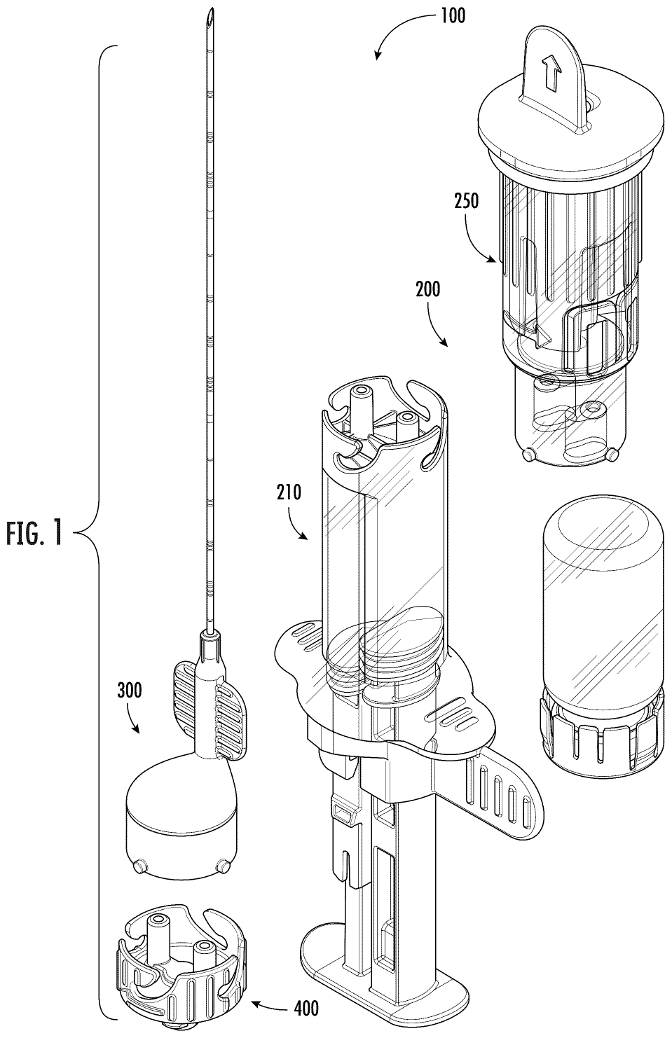

Turning now to the drawings, an example of an embodiment of a combining and/or delivery system 100 formed in accordance with various principles of the present disclosure is illustrated in FIG. 1. The combining and/or delivery system 100 includes a multi-chamber system 200 and an injection system 300. The multi-chamber system 200 may be used to transport components of an injectable material to a facility/location at which the injectable material is to be delivered into a patient. For the sake of convenience, and without intent to limit, reference may be made to delivery by injection into the patient (e.g., the injectable material is delivered to the patient by being injected into the patient), although the present disclosure need not be so limited. The material combined and/or delivered by a combining and/or delivery system 100 of the present disclosure may be any other type of material to be administered to a patient, such as other than by injection. The injectable material may, as described above, include two or more components which are to remain separate until the time of the procedure. Once a medical professional is ready to inject the injectable material into the patient, the components of the injectable material are combined by the combining and/or delivery system 100 to be combined into (e.g., to react with each other to form) a compound material having the desired characteristics for being delivered to and/or deposited at a target site by the combining and/or delivery system 100. The multi-chamber system 200 may be fluidly coupled with the injection system 300 of the combining and/or delivery system 100 to combine the injectable material and/or to deliver the injectable material to the patient. The injection system 300 may be usable independently of the multi-chamber system 200. An optional adaptor system 400 may be included with the combining and/or delivery system 100 to adapt the injection system 300 for use with an off-the-shelf (e.g., pre-existing, separately sold, commercially available, etc.) device used to perform a procedure, treatment, pretreatment, etc., prior to or after delivery of the injectable material to the target site, as described in further detail below. It will be appreciated that features of the multi-chamber device 210 of the multi-chamber device 210 facilitating coupling of the multi-chamber device 210 with the injection system 300 may also facilitate coupling of the multi-chamber device 210 with other devices or systems, such as other devices of the multi-chamber system 200, such as a vial adaptor 250. Likewise, it will be appreciated that features of the injection system 300 facilitating coupling of the injection system 300 with the multi-chamber device 210 may also facilitate coupling of the injection system 300 with other devices or systems, such as the adaptor system 400.

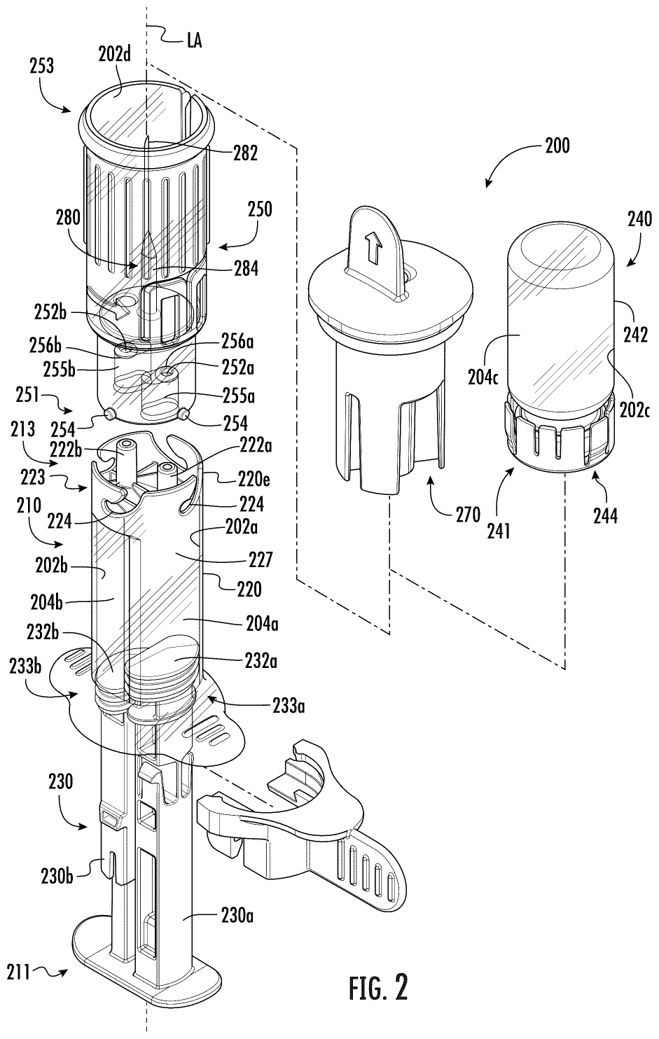

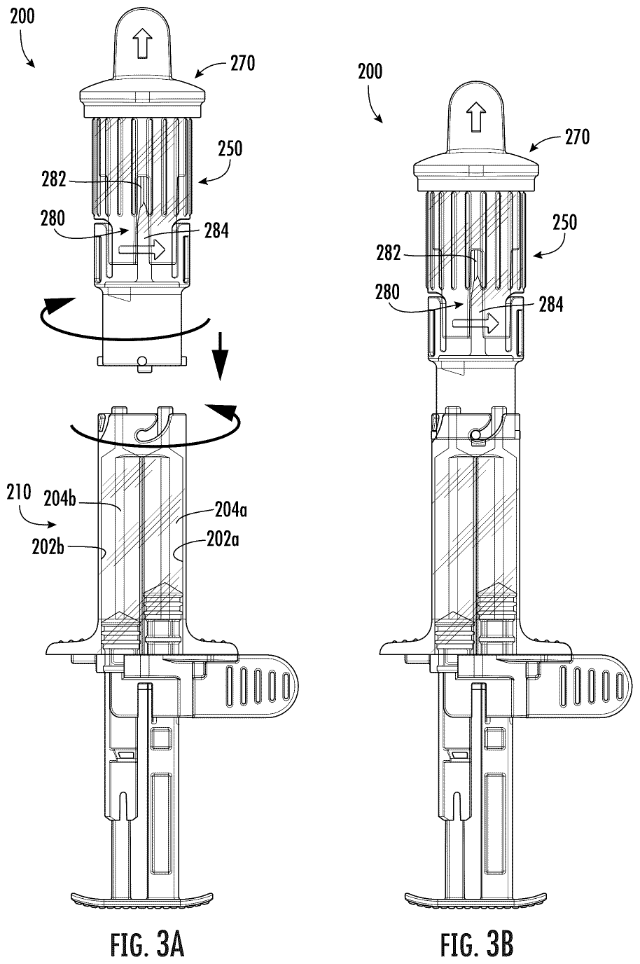

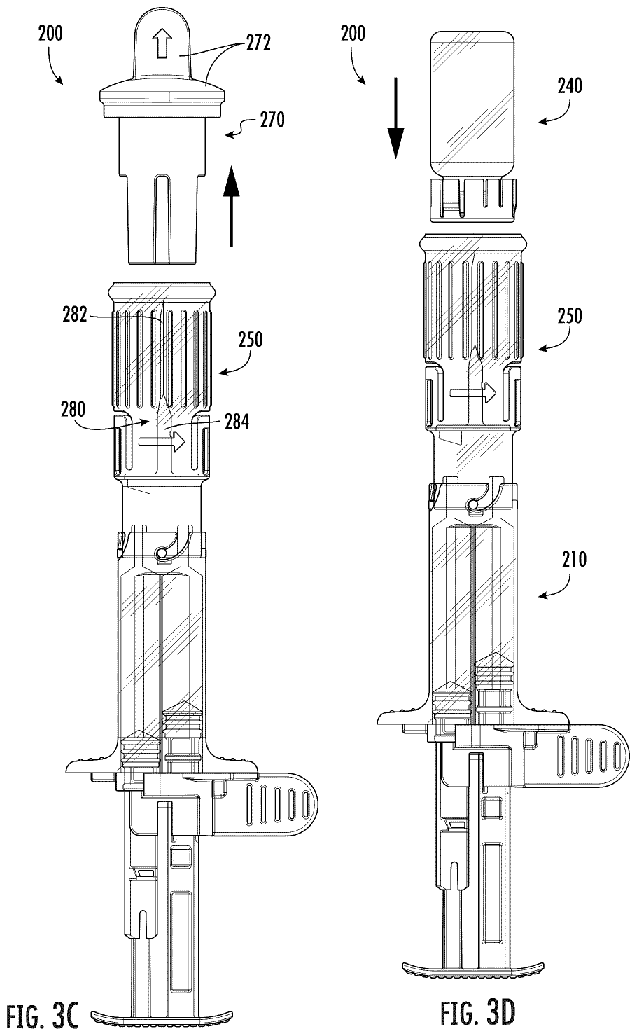

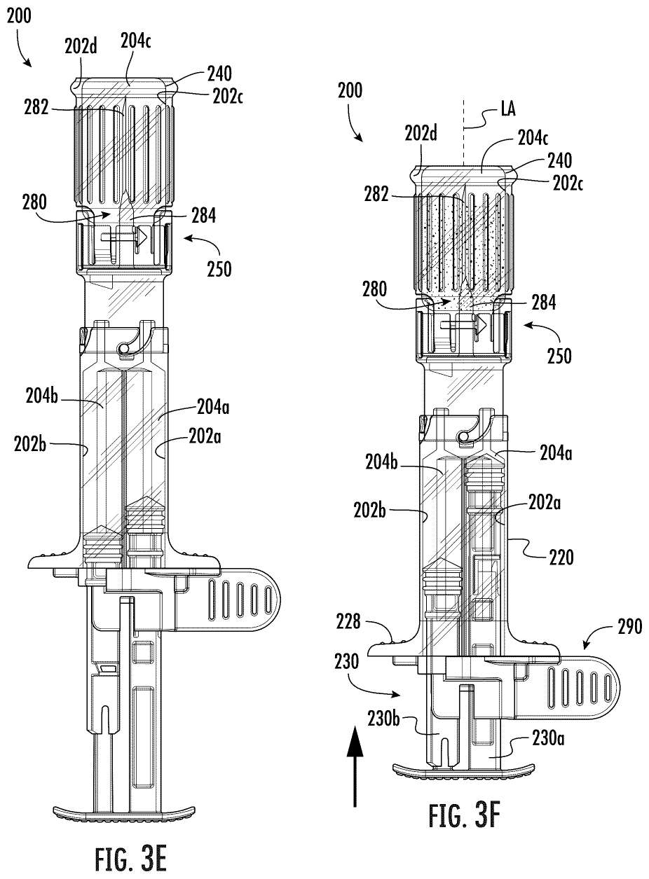

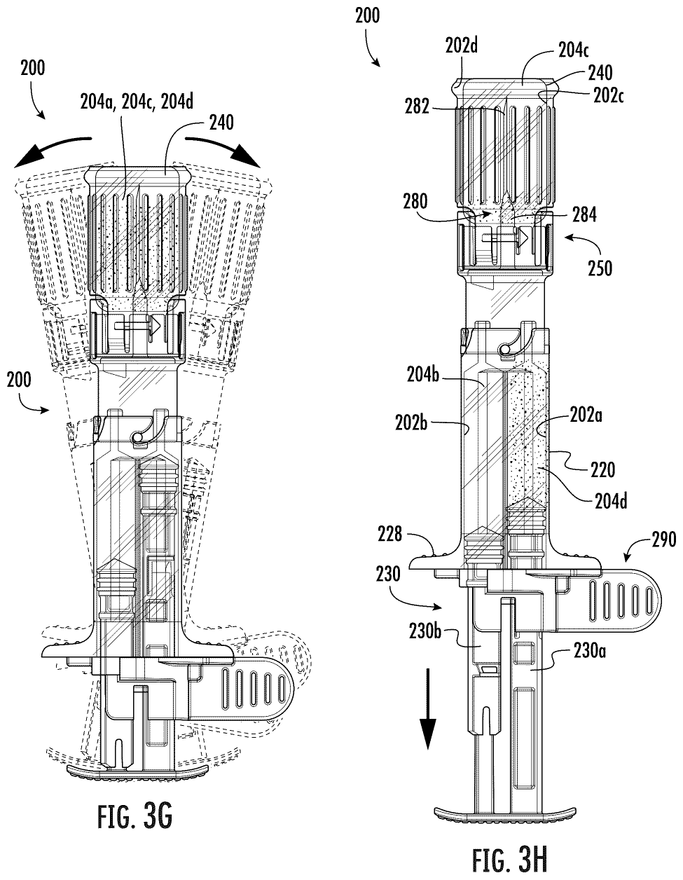

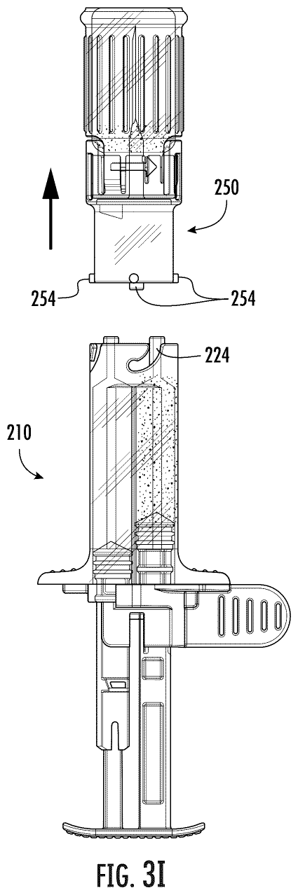

Various devices or elements of the example of an embodiment of a multi-chamber system 200 illustrated in FIG. 1 may be appreciated with reference to FIG. 2. The multi-chamber system 200 includes one or more devices each having at least one chamber for containing a component of an injectable material to be delivered to a patient. In the example of an embodiment illustrated in FIG. 2, the multi-chamber system 200 includes a multi-chamber device 210 having a barrel 220 defining a first chamber 202a and a second chamber 202b of the multi-chamber system 200. In some embodiments, the chambers 202a, 202b have substantially symmetrical cross-sectional shapes, such as half-circles if within a barrel 220 having a substantially circular cross-section, although the configurations need not be so limited. For instance, the chambers 202a, 202b may have different volumes, cross-sectional shapes, etc., such as may be dictated by the properties of the components to be contained therein. The first chamber 202a is defined to be separate and fluidly-isolated from the second chamber 202b. For instance, the contents of the first chamber 202a, e.g., a first component 204a of an injectable material, may react with the contents of the second chamber 202b, e.g., a second component 204b of an injectable material, when combined. In some embodiments, the first component 204a is combined with a third component 204c of the injectable material, such as to form a precursor 204d (such as illustrated with reference to FIG. 3G and FIG. 3H), and the second component 204b may react with the precursor 204d (e.g., to accelerate reaction of the first component 204a and the third component 204c). It may be desirable to keep these components separate until ready for delivery to a patient. To avoid unintentional premature combination, the contents of the chambers 202a, 202b are maintained separate from each other by the fluid isolation of the chambers 202a, 202b. When (e.g., only when) a medical professional is ready to deliver the injectable material (e.g., as a combined injectable material), the components within the chambers 202a, 202b may be combined and allowed to react with each other. The various devices, systems, and method of the present disclosure prevent unintentional combination of the first component 204a and the second component 204b, such as before the medical professional is ready to deliver the injectable material (to be formed from the first component 204a and the second component 204b) to a patient as a combined injectable material. Additionally or alternatively, the various devices, systems, and method of the present disclosure facilitate case and accuracy of combining of components to form an injectable material at the appropriate time.

The example of an embodiment of a multi-chamber device 210 illustrated in FIG. 2 further includes a plunger assembly 230 extending proximally from the barrel 220 (towards the proximal end 211 of the multi-chamber device 210). The plunger assembly 230 is movable, such as axially/longitudinally slidable (e.g., along the longitudinal axis LA), with respect to the barrel 220 to move (e.g., eject) materials out of and/or to move (e.g., aspirate) materials into chambers within the barrel 220. In the illustrated example of an embodiment, the plunger assembly 230 has a first plunger rod 230a movable/slidable with respect to the first chamber 202a and a second plunger rod 230b movable/slidable with respect to the second chamber 202b. In the illustrated example of an embodiment, distal movement of the plunger rods 230a, 230b moves materials out of the first chamber 202a and the second chamber 202b, respectively, via a first nozzle 222a and a second nozzle 222b, respectively, extending distally from the distal end 223 of the barrel 220. Conversely, proximal movement of the plunger rods 230a, 230b moves materials into the first chamber 202a and the second chamber 202b, respectively, via the first nozzle 222a and the second nozzle 222b, respectively. It will be appreciated that the plunger rods 230a, 230b may move independently of each other. Optionally, the first plunger rod 230a has a plunger stopper 232a at the distal end 233a thereof, and/or the second plunger rod 230b has a plunger stopper 232b at the distal end 233b thereof. The plunger stoppers 232a, 232b may be formed of an appropriate sealing material (e.g., an elastomeric material) known to those of ordinary skill in the art for retaining the first component 204a within the first chamber 202a and the second component 204b within the second chamber 202b.

The multi-chamber system 200 optionally includes a third chamber 202c for containing an additional, third component 204c. The third component 204c may be combinable with the first component 204a contained within the first chamber 202a. The third chamber 202c optionally is defined in a separate device 240, formed and provided separately from the multi-chamber device 210, such as illustrated in FIG. 2. For the sake of convenience, and without intent to limit, the separate device is referenced herein as a vial 240, having a vial body 242 defining the third chamber 202c (which may alternately be referenced herein as a vial chamber 202c), and a vial cap 244 closing an open end 241 of the vial body 242 to contain (e.g., seal) the third component 204c within the third chamber 202c. The vial cap 244 may include a plug or stopper formed of a suitable material known to those of ordinary skill in the art as capable of scaling the third component 204c within the third chamber 202c defined within the vial 240 and optionally also allowing access therethrough, as described in further detail below. For example, the stopper may be formed of an elastic material capable of being pierced to allow fluid communication therethrough, such as described in further detail below.

A vial adaptor 250 is configured to facilitate holding/coupling of the vial 240 with respect to the multi-chamber device 210 to transfer materials therebetween, in a manner described in further detail below. The example of an embodiment of a vial adaptor 250 illustrated in FIG. 2 defines a fourth chamber 202d of the multi-chamber system 200 opening to the distal end 253 of the vial adaptor 250. The fourth chamber 202d may be configured as a vial-receiving chamber configured to receive the vial 240 and to hold the vial 240 in alignment with the multi-chamber device 210 so that the multi-chamber device 210 and the vial 240 may be fluidly coupled for transferring and/or exchanging material therebetween. For instance, the vial adaptor 250 may facilitate alignment and/or fluid sealing between the vial adaptor 250 and the multi-chamber device 210 so that materials may be transferred between a vial 240 (within the vial adaptor 250) and the multi-chamber device 210 without leakage, and/or without significant manipulation of components of the combining and/or delivery system 100. More particularly, the vial adaptor 250 facilitates fluid coupling of a first chamber 202a of a multi-chamber device 210 formed in accordance with various principles of the present disclosure with a separate chamber 202c, and the combination of the components 204a, 204c contained within the chambers 202a, 202c. The vial adaptor 250 may include a first nozzle port 252a positioned and configured to be fluidly coupled with (e.g., to receive and to be in fluid communication with) the first nozzle 222a of the barrel 220 of the multi-chamber device 210, and a second nozzle port 252b positioned and configured to be fluidly coupled with (e.g., to receive and to be in fluid communication with) the second nozzle 222b of the barrel 220 of the multi-chamber device 210. In accordance with various principles of the present disclosure, the first nozzle port 252a includes a first nozzle pocket 255a sized, shaped, configured, and/or dimensioned to facilitate engagement and proper seating of the first nozzle 222a of the barrel 220 of the multi-chamber device 210 with the first nozzle port 252a of the vial adaptor 250, as discussed in further detail below. Likewise, in accordance with various principles of the present disclosure, the second nozzle port 252b includes a second nozzle pocket 255b positioned, sized, shaped, configured, and/or dimensioned to facilitate engagement and proper seating of the second nozzle 222b of the barrel 220 of the multi-chamber device 210 with the second nozzle port 252b of the vial adaptor 250, as discussed in further detail below.

The vial adaptor 250 and the multi-chamber device 210 are configured to facilitate secure manual fluid coupling and alignment of the vial adaptor 250 with the multi-chamber device 210, such as to facilitate fluid coupling of the multi-chamber device 210 with a vial 240 positioned within the vial adaptor 250. For instance, the barrel 220 of the multi-chamber device 210, and the vial adaptor 250 may each optionally include one or more (e.g., 1-4, or more) corresponding engaging elements 224, 254, respectively, configured to engage with one another in a manner which facilitates manual coupling of the multi-chamber device 210 and the vial adaptor 250. More particularly, in the example of an embodiment of a multi-chamber system 200 illustrated in FIG. 2, the distal end 223 of the barrel 220 of the multi-chamber device 210 includes one or more (e.g., 1-4, or more) vial-adaptor-engaging elements 224, and the proximal end 251 of the vial adaptor 250 includes one or more (e.g., 1-4, or more) barrel-engaging elements 254. Even more particularly, the example of an embodiment of a barrel 220 of a multi-chamber device 210 has a distal extension 220c on which the one or more vial-adaptor-engaging elements 224 are provided. The distal extension 220e may be sized, shaped, configured, and/or dimensioned to receive a proximal end 251 of the vial adaptor 250.

The one or more engaging elements 224 on the barrel 220 and the one or more barrel-engaging elements 254 on the vial adaptor 250 are configured to engage with one another in a manner facilitating ready, simplified manual coupling of the proximal end 251 of the vial adaptor 250 with the distal end 213 of the multi-chamber device 210. For instance, in the example of an embodiment of a multi-chamber system 200 illustrated in FIG. 2, the one or more engaging elements 224 of the barrel 220 of the multi-chamber device 210 receive and interlock with one or more barrel-engaging elements 254 on the vial adaptor 250. The barrel-engaging elements 254 on the vial adaptor 250 may be configured to readily be initially engaged with respect to, and/or to remain engaged with, and/or to be readily disengaged from the engaging elements 224 on the barrel 220. For instance, the engaging elements 224 of the barrel 220 may be in the form of slots, and the barrel-engaging elements 254 on the vial adaptor 250 may be in the form of radially-outwardly extending bosses, projections, pins, nubs, protrusions, etc. (referenced herein as projections for the sake of convenience and without intent to limit) sized, shaped, configured, and/or dimensioned to engage and move with respect to the engaging elements 224 on the multi-chamber device 210. In the example of an embodiment illustrated in FIG. 2, the engaging elements 224 on the multi-chamber device 210 are formed as slots extending through the barrel distal extension 220e from the interior surface 225 of the barrel distal extension 220e to the exterior surface 227 of the barrel distal extension 220e. However, in some embodiments, slots forming the vial-adaptor-engaging elements 224 extend from the interior surface 225 of the barrel distal extension 220e only partially through the barrel distal extension 220e (i.e., not extending through to the exterior surface 227 of the barrel distal extension 220e. Such configuration of vial-adaptor engaging elements 224 and barrel-engaging elements 254 allows a relatively simple (e.g., a bayonet-type) connection between the multi-chamber device 210 and the vial adaptor 250, facilitating ready engagement and/or disengagement of the vial adaptor 250 with respect to the multi-chamber device 210. For instance, the vial adaptor 250 and the multi-chamber device 210 may be brought together generally axially to engage the vial-adaptor-engaging elements 224 of the multi-chamber device 210 with the barrel-engaging elements 254 of the vial adaptor 250, and then rotating the multi-chamber device 210 and the vial adaptor 250 with respect to each other to cause the barrel-engaging elements 254 of the vial adaptor 250 to interlock with the vial-adaptor-engaging elements 224 of the barrel 220 to hold the vial adaptor 250 and multi-chamber device 210 in place with respect to each other. Such configuration allows the multi-chamber device 210 to be readily engaged/coupled with the vial adaptor 250 in a manner inhibiting axial separation of these components once coupled, and allowing ready disengaging/decoupling, such as with reverse relative movements of the multi-chamber device 210 and vial adaptor 250, as described below in further detail with reference to FIGS. 3A-3I. It will be appreciated that reverse arrangements of engagement elements of the multi-chamber device 210 and/or the vial adaptor 250 are within the scope of the present disclosure. For instance, radially extending projections may be provided along a distal end 213 the multi-chamber device 210 for engaging within slots or grooves defined along a proximal end 251 of the vial adaptor 250. Other configurations of engaging elements, such as known those of ordinary skill in the art, are within the scope and spirit of the present disclosure, the present disclosure not being limited by the particular configurations illustrated in the drawings.

As noted above, coupling of the multi-chamber device 210 and the vial adaptor 250 with the assistance of their respective engaging elements 224, 254 may be accomplished by generally axial as well as by rotational relative movements of the multi-chamber device 210 and the vial adaptor 250. In some embodiments, engaging elements formed in accordance with various principles of the present disclosure in the shape of slots have a generally axially-extending segment 224a and a generally circumferentially-extending segment 224c. As described in greater detail below, the generally axially-extending slot segment allows the multi-chamber device 210 and the vial adaptor 250 to be brought together generally axially to bring their respective engaging elements 224, 254 into initial engagement with one another. The generally circumferentially-extending slot segment allows relative rotation of the multi-chamber device 210 and the vial adaptor 250 to engage their respective engaging elements 224, 254 in a manner which inhibits axial separation of the engaging elements 224, 254 and thus the multi-chamber device 210 and the vial adaptor 250. In some embodiments, the generally circumferentially-extending slot segment ends (i.e., opposite the end adjacent the generally axially-extending segment 224a) in a generally axially-extending locking section 2240. A biasing element may be provided to bias the multi-chamber device 210 and the vial adaptor 250 apart from each other to cause the engaging element in the form of a projection to extend into the generally axially-extending locking section 2240 to inhibit unintended relative rotation of the multi-chamber device 210 and the vial adaptor 250 (which may lead to decoupling of the multi-chamber device 210 and the vial adaptor 250). In some embodiments, the generally axially-extending locking section 2240 extends towards the one of the multi-chamber device 210 or the vial adaptor 250 carrying the engaging elements configured as projections so that biasing the multi-chamber device 210 and the vial adaptor 250 apart causes the engaging elements configured as projections to extend into the generally axially-extending locking section to inhibit unintended relative rotation of the multi-chamber device 210 and the vial adaptor 250. It will be appreciated that a reverse configuration is within the scope of the present disclosure, with the generally axially-extending locking section 224l extending away from the one of the multi-chamber device 210 or the vial adaptor 250 carrying the engaging elements configured as projections so that biasing the multi-chamber device 210 and the vial adaptor 250 together causes the engaging elements configured as projections to extend into the generally axially-extending locking section 2240 to inhibit unintended relative rotation of the multi-chamber device 210 and the vial adaptor 250.

The vial adaptor 250 may be coupled with the multi-chamber device 210 with a protective cap 270 positioned within the fourth chamber 202d (instead of and prior to insertion of the vial 240 into the fourth chamber 202d of the vial adaptor 250). The protective cap 270 may be configured to protect or shield components of a fluid exchange device 280 extending within the fourth chamber 202d of the vial adaptor 250 (such as a needle 282 and fluid exchange spike 284, as illustrated in FIG. 3A, FIG. 3B, and FIG. 3C). The fluid exchange device 280 is configured to fluidly communicate the first chamber 202a within the barrel 220 of the multi-chamber device 210 with the third chamber 202c within the vial 240, once the vial 240 is positioned within the vial chamber 202c, and the needle 282 is extended into the vial 240 (such as illustrated in FIG. 3E, FIG. 3F, and FIG. 3H). The protective cap 270 may also protect the vial adaptor 250 from entry of unwanted matter or debris from entering the fourth chamber 202d.

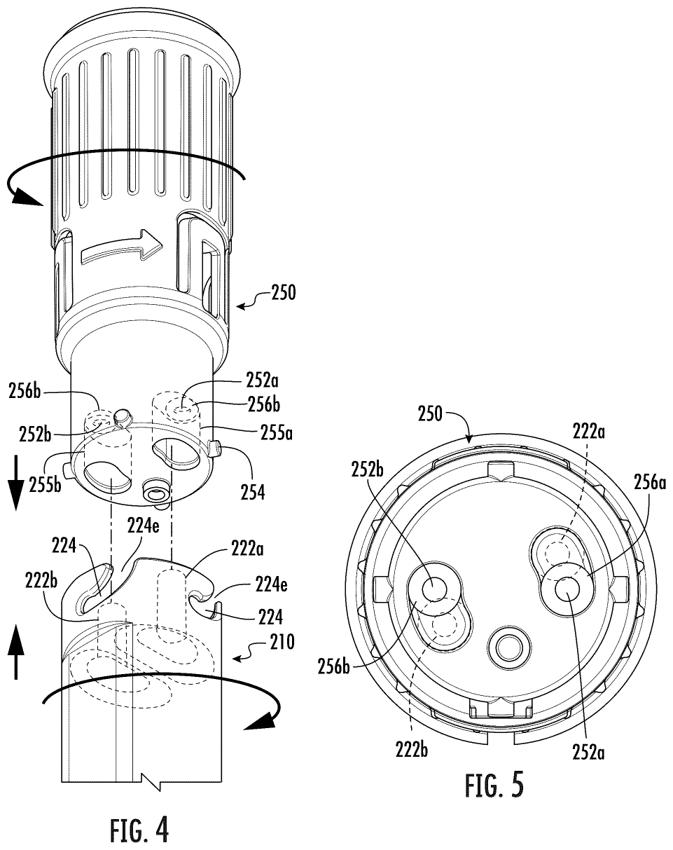

The various interactions of the various components of a multi-chamber system 200 formed in accordance with various principles of the present disclosure and illustrated in FIG. 2 may be appreciated with reference to FIGS. 3A-3I. The multi-chamber device 210 and the vial adaptor 250 are illustrated in FIG. 3A in position to be assembled together for delivery of the first component 204a and the second component 204b (within respective chambers 202a, 202b defined within the barrel 220 of the multi-chamber device 210), ready for combining the first component 204a with a third component 204c before delivery to a patient. As may be appreciated with reference to FIG. 4, showing a bottom perspective view of the vial adaptor 250 and a distal portion of the multi-chamber device 210, positioned relative to each other as illustrated in FIG. 3A, the nozzles 222a, 222b of the multi-chamber device 210 are positioned to be inserted into the nozzle pockets 255a, 255b of the vial adaptor 250 upon bringing together the multi-chamber device 210 and the vial adaptor 250, such as illustrated in FIG. 3B. However, the proximal end view of the vial adaptor 250 in FIG. 5 shows, superimposed in phantom, the positions of the nozzles 222a, 222b with respect to the nozzle ports 252a, 252b when the multi-chamber device 210 and the vial adaptor 250 are oriented with respect to each other as in FIG. 4. As may be appreciated, the nozzles 222a, 222b are not aligned with the nozzle ports 252a, 252b. The multi-chamber device 210 and the vial adaptor 250 must be rotated with respect to each other along the circumferentially-extending directional arrows illustrated in FIG. 3A and FIG. 4 for the nozzles 222a, 222b to be aligned and seated with respect to the nozzle ports 252a, 252b, respectively, as well as with the seals 256a, 256b, if provided aligned with the nozzle ports 252a, 252b.

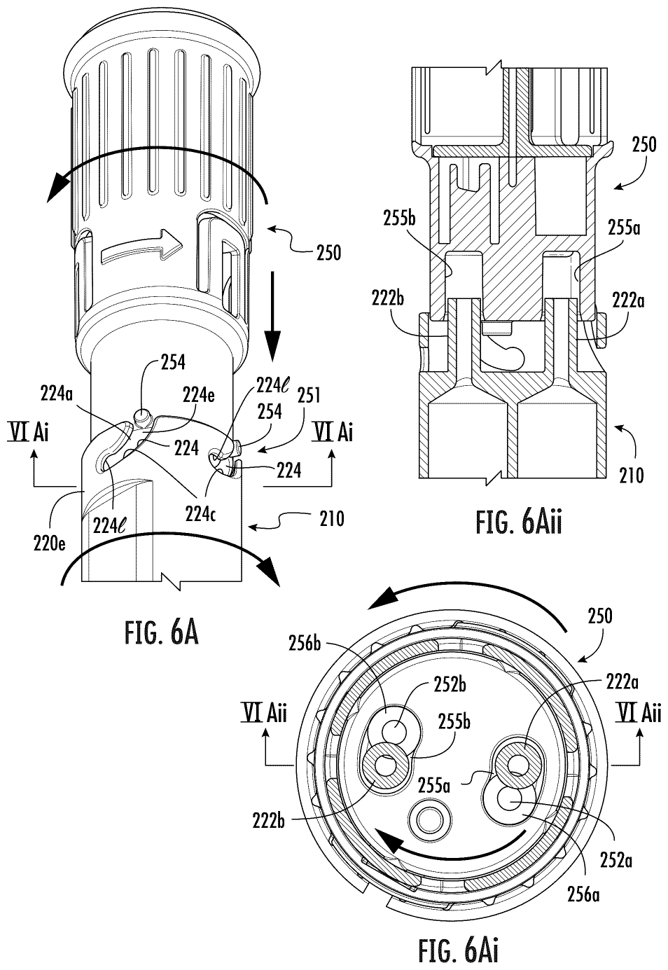

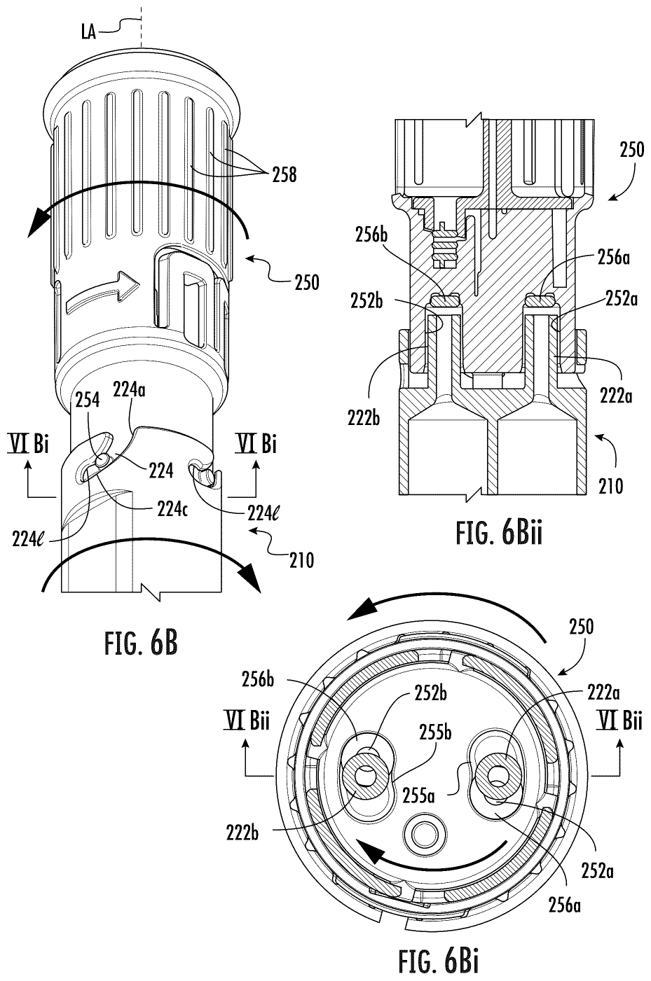

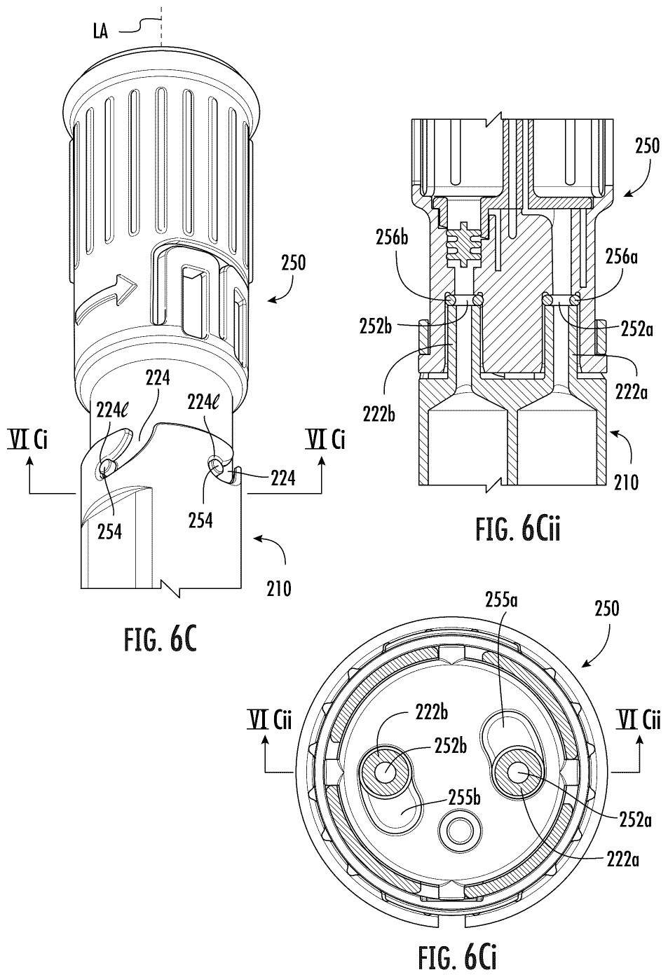

To prepare the multi-chamber system 200 for combining the first component 204a with a third component 204c (such as delivered within the optional vial 240), the vial adaptor 250 and the multi-chamber device 210 are brought together, from positions such as illustrated in FIG. 3A and FIG. 4, to be fluidly coupled together such as illustrated in FIG. 3B. The relative movements of the vial adaptor 250 and the multi-chamber device 210 to fluidly couple together the vial adaptor 250 and the multi-chamber device 210 are illustrated in further detail in FIG. 6A, FIG. 6B, and FIG. 6C. As illustrated in FIG. 6A, once the vial adaptor 250 and multi-chamber device 210 have been brought together generally axially (along the longitudinal axis LA), the barrel-engaging elements 254 of the vial adaptor 250 may be positioned to enter the vial-adaptor-engaging elements 224 via the entry openings 224e thereof. In some embodiments, a proximal end 251 of the vial adaptor 250 is partially inserted into the barrel distal extension 220e and rotated (with the barrel distal extension 220e optionally providing some guidance) to align the barrel-engaging elements 254 with the entry opening 224e of the vial-adaptor-engaging elements 224.

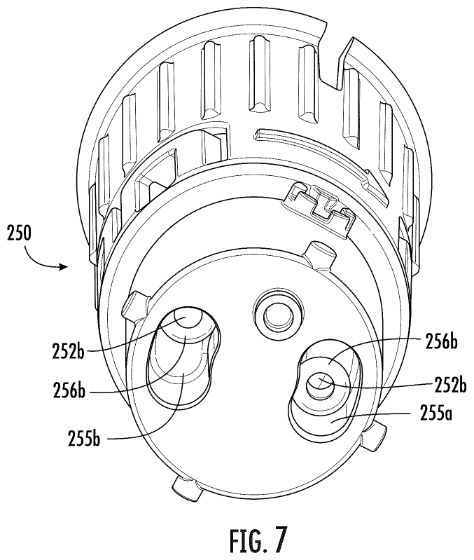

As may be appreciated with reference to the cross-sectional view of FIG. 6Ai, along line VIAi-VIAi in FIG. 6A, in the position illustrated in FIG. 6A, the nozzles 222a, 222b of the multi-chamber device 210 are not aligned with the nozzle ports 222a, 222b of the vial adaptor 250. Nonetheless, as may be appreciated with reference to the cross-sectional view of FIG. 6Aii, along line VIAii-VIAii in FIG. 6B, in the position illustrated in FIG. 6A, the nozzles 222a, 222b of the multi-chamber device 210 are positioned within the nozzle pockets 255a, 255b, such that the nozzle pockets 255a, 255b may align and/or guide the nozzles 222a, 222b axially/or rotationally with respect to the nozzle ports 252a, 252b of the vial adaptor 250 as the barrel-engaging elements 254 are rotated toward the locking section 2240 of the vial-adaptor-engaging elements 224. For instance, as may be appreciated with reference to the proximal perspective view of the vial adaptor 250 illustrated in FIG. 7, as well as with reference to FIG. 4, FIG. 6Bi, FIG. 6Bii, FIG. 6Ci, FIG. 6Cii, the illustrated examples of embodiments of nozzle pockets 255a, 255b are positioned, sized, shaped, configured, and/or dimensioned to facilitate coupling of the vial adaptor 250 and the multi-chamber device 210 with the use of engaging elements 224, 254 which involve relative axial and rotational movement of the vial adaptor 250 and the multi-chamber device 210. More particularly, the nozzle pockets 255a, 255b extend generally axially and generally circumferentially with respect to the longitudinal axis LA of the vial adaptor 250 and may be considered to guide the nozzles 222a, 222b axially and/or rotationally with respect to the nozzle ports 252a, 252b, respectively, such as to be in alignment and/or be seated with respect thereto. If seals 256a, 256b are provided within the vial adaptor 250, then the nozzle pockets 255a, 255b may also guide the nozzles 222a, 222b of the multi-chamber device 210 into alignment with, to be seated with respect to, the seals 256a, 256b of the vial adaptor 250.

Further generally axial relative movement of the vial adaptor 250 and the multi-chamber device 210 together, from the positions illustrated in FIG. 6A to the positions illustrated in FIG. 6B, bring the barrel-engaging elements 254 of the vial adaptor 250 into the generally axially-extending segments 224a of the engaging elements 224 of the multi-chamber device 210. The vial adaptor 250 and the multi-chamber device 210 may then be moved axially together further. As the vial adaptor 250 and the multi-chamber device 210 are moved together axially, the barrel-engaging elements 254 extend into the generally circumferentially-extending segments 224c of the engaging elements 224, and are moved toward the locking sections 2240. The vial adaptor 250 and the multi-chamber device 210 thus are also rotated with respect to each other to be moved from the position illustrated in FIG. 6A to a position such as illustrated in FIG. 6B. Optionally the vial adaptor 250 may be provided with surface features 258 (such as ribs, grooves, bumps, dimples, etc.) enhancing grasping thereof, in a manner known to those of ordinary skill in the art. As may be appreciated with reference to FIG. 6Bi, taken along line VIBi-VIBi in FIG. 6B, such further relative rotation of the vial adaptor 250 and the multi-chamber device 210 moves the nozzles 222a, 222b closer to the nozzle ports 252a, 252b. And, as may be appreciated with reference to FIG. 6Bii, taken along line VIBii-VIBii in FIG. 6Bi, the nozzles 222a, 222b also are brought closer to optional seals 256a, 256b seated with respect to the nozzle ports 252a, 252b, respectively.

After further relative rotation of the vial adaptor 250 and the multi-chamber device 210 in the directions indicated in FIG. 6A and FIG. 6B, the barrel-engaging elements 254 of the vial adaptor 250 reach the locking sections 2240 of the engaging elements 224 as illustrated in FIG. 6C. In the configuration illustrated in FIG. 6C, the multi-chamber device 210 and the vial adaptor 250 are fluidly coupled, with the nozzles 222a, 222b of the multi-chamber device 210 seated with respect to the nozzle ports 252a, 252b of the vial adaptor 250, as may be appreciated with reference to FIG. 6Ci, taken along line VICi-VICi in FIG. 6C, and FIG. 6Cii, taken along line VICii-VICii in FIG. 6Ci. Optionally, at least one of the locking sections 2240 extends generally axially, and thus transverse to the generally circumferentially-extending segments 224c of the engaging element 224. As such, a barrel-engaging element 254 seated within the at least one locking section 2240 resists inadvertent relative rotation of the vial adaptor 250 and the multi-chamber device 210 in a direction which would bring the barrel-engaging element 254 out of its position with respect to the at least one locking section 2240. The at least one locking section 2240 thus maintains the fluid coupling of the multi-chamber device 210 and the vial adaptor 250 with the nozzles 222a, 222b seated with respect to the nozzle ports 252a, 252b, respectively. Optional seals 256a, 256b, seated with respect to the nozzle ports 252a, 252b, respectively, such as to fluidly seal the connection between the nozzles 222a, 222b and the corresponding nozzle ports 252a, 252b, may provide a biasing force moving the multi-chamber device 210 and the vial adaptor 250 apart to retain a barrel-engaging element 254 seated with respect to the at least one locking section 224l to inhibit inadvertent relative rotation of the multi-chamber device 210 and the vial adaptor 250. Additionally or alternatively, compression of optional seals 256a, 256b may exert an axial force increasing friction resisting relative rotation of the multi-chamber device 210 and the vial adaptor 250 in a direction which could lead to decoupling of the multi-chamber device 210 and the vial adaptor 250

Returning to FIG. 3B, the optional protective cap 270 (illustrated in isolation in FIG. 2) is illustrated positioned within the fourth chamber 202d of the vial adaptor 250. Once the vial adaptor 250 is fluidly coupled with the multi-chamber device 210 (with the nozzles 222a, 222b of the multi-chamber device 210 seated with respect to the nozzle ports 252a, 252b of the vial adaptor 250), such as illustrated in FIG. 3B (such as upon the stages of relative movement of the multi-chamber device 210 and vial adaptor 250 described above with reference to FIG. 6A, FIG. 6B, and FIG. 6C), the protective cap 270 is separated from the vial adaptor 250 (e.g., removed from the fourth chamber 202d), as illustrated in FIG. 3C. Optionally, the protective cap 270 includes a grasping section 272, such as a radially-outwardly projecting flange and/or an axially extending flange, configured to facilitating grasping and pulling on the protective cap 270 to remove the protective cap 270 from the vial-receiving fourth chamber 202d defined in the vial adaptor 250. Optionally the grasping section 272 may be provided with surface features such as bumps, ribs, grooves, etc., enhancing grasping thereof, in a manner known to those of ordinary skill in the art.

The vial 240 may then be aligned with the vial adaptor 250 for insertion into the fourth chamber 202d, as illustrated in FIG. 3D. In FIG. 3E, the vial 240 is illustrated positioned within the fourth chamber 202d of the vial adaptor 250, with the fluid exchange device 280 fluidly communicating the first chamber 202a of the multi-chamber device 210 with the third chamber 202c within the vial 240. The fluid exchange device 280 may be considered to include components of the multi-chamber device 210, the vial adaptor 250, and the vial 240 configured and engaged to facilitate combining of the first component 204a, contained within the first chamber 202a defined in the barrel 220 of the multi-chamber device 210, with the third component 204c, contained within the third chamber 202c defined in the vial 240. In the example of an embodiment of a multi-chamber system 200 as illustrated in FIGS. 3A-3F, the fluid exchange device 280 includes a piercing element, such as a needle 282, extending from a fluid exchange spike 284 configured to fluidly communicate the first chamber 202a and the third chamber 202c, such as illustrated in FIG. 3E and FIG. 3F.

Once the vial 240 is seated within the fourth chamber 202d of the multi-chamber system 200 defined by the vial adaptor 250, and the third chamber 202c defined within the vial 240 is in fluid communication with the first chamber 202a defined within the barrel 220 of the multi-chamber device 210, such as via the fluid exchange device 280, such as illustrated in FIG. 3E, the plunger assembly 230 may be advanced distally with respect to the barrel 220 of the multi-chamber device 210, as illustrated in FIG. 3F. A finger grip 228 (e.g., one or more radially-outwardly extending flanges) may be provided on the barrel 220 to facilitate grasping and/or holding of the barrel 220 to move the plunger assembly 230 relative to the barrel 220. As may be appreciated with reference to FIG. 3F, the plunger assembly 230 may be configured such that the first plunger rod 230a and the second plunger rod 230b are independently advanceable. More specifically, the first plunger rod 230a and the second plunger rod 230b do not necessarily advance together. Such configuration of the plunger assembly 230 allows the first plunger rod 230a to eject the first component 204a out of the first chamber 202a and into the vial 240 without the second plunger rod 230b ejecting the second component 204b out of the second chamber 202b. For instance, the first plunger rod 230a may extend the full, or close to the full, longitudinal extent of the first chamber 202a (along a longitudinal axis LA, such as of or substantially parallel to a longitudinal axis of the barrel 220) to expel all or most of the first component 204a out from the first chamber 202a and to inject the first component 204a into the third chamber 202c for combination with the third component 204c therein.

Once the first component 204a has been injected into the third chamber 202c, the multi-chamber system 200 may be shaken, such as schematically illustrated in FIG. 3G, such as to facilitate mixing of the first component 204a and the third component 204c within the vial 240 to form the precursor 204d. The combined first component 204a and third component 204c are then aspirated back into the first chamber 202a by retracting the plunger assembly 230 proximally, as illustrated in FIG. 3H. For the sake of convenience, and without intent to limit, the combination of the first component 204a and the third component 204c is referenced herein as a precursor 204d. It will be appreciated that in some instances, the entire content of the vial 240 (the combined first component 202a and third component 202c) is not aspirated out of the vial. 240. In some embodiments, the second component 204b (within the second chamber 202b) may react with the precursor 204d if combined therewith (e.g., the second component 204b may be an accelerant for completing a reaction between the first component 204a and the second component 204b). As such, as described above with respect to first component 204a and the second component 204b, the precursor 204d and the second component 204b may be maintained separate, such as by fluid isolation of the first chamber 202a and the second chamber 202b, until the user is ready for intentional combination of such components (e.g., when ready for delivery to a patient). Also, an optional retainer 290 (illustrated in isolation in FIG. 2) is illustrated in FIG. 3A-FIG. 3I as coupled with the plunger assembly 230 (e.g., mounted thereon) to limit movement of the plunger assembly 230 with respect to the barrel 220 to prevent inadvertent ejection of materials from the multi-chamber device 210.

Once the combined first component 204a and third component 204c have been aspirated into the first chamber 202a, the vial 240 and the vial adaptor 250 are withdrawn from the multi-chamber device 210, as illustrated in FIG. 3I. Relative movement (rotational and generally axial movement) of the multi-chamber device 210 and the vial adaptor 250 with respect to each other in a manner opposite to the movements described above with reference to FIG. 6A, FIG. 6B, and FIG. 6C allows the barrel-engaging elements 254 of the vial adaptor 250 and the engaging elements 224 of the multi-chamber device 210 to disengage from one another to release the vial adaptor 250 from the multi-chamber device 210, such as illustrated in FIG. 3I. It will be appreciated that such configuration of engaging elements 224, 254 as described above provides a relatively simple engagement and alignment of the multi-chamber device 210 and the vial adaptor 250 for combination of the first component 204a and the third component 204c within the vial 240. It will further be appreciated that such configuration of engaging elements 224, 254 as described above provides a relatively simple disengagement of the vial adaptor 250 from the multi-chamber device 210. The multi-chamber device 210, with the first chamber 202a of the multi-chamber device 210 now containing the precursor 204d, and the second chamber 202b of the multi-chamber device 210 containing a second component 204b (such as an accelerant) combinable with the precursor 204d, is now ready to deliver the injectable materials contained within the first chamber 202a and the second chamber 202b to the patient.

It will be appreciated that although the example of an embodiment of a multi-chamber system 200 illustrated in FIG. 2 has a multi-chamber device 210 configured to deliver a first component separate from a second component for combining upon delivery to a patient, various principles of the present disclosure are applicable to various first devices with a nozzle configured to eject or aspirate an injectable material (with respect to a chamber in the first device) and/or various second devices with ports with respect to which a nozzle may be seated and through which an injectable material may be delivered (e.g., from or into the first device, to a patient, into or from another device, etc.). For instance, various principles of the present disclosure may be applied to various devices defining a material chamber therein and having at least one nozzle fluidly communicating with the material chamber and through which material is ejected and/or aspirated. Additionally or alternatively, various principles of the present disclosure may be applied to devices having a port with respect to which a nozzle is seated and through which material is aspirated or ejected with respect to the nozzle. For instance, the injectable material transfer device may be a single-chamber device, such as known to those of ordinary skill in the art, similar to the multi-chamber device 210, but with only one chamber defined within the barrel 220 (illustration thereof not being considered necessary for full understanding thereof by those of ordinary skill in the art). Components within the single-chamber device may be combined with components within a separately-provided chamber (e.g., an additional component provided in a distinct and/or separate device with a chamber for such additional component, such as a vial separate from the single-chamber device). Alternatively, the multi-chamber device 210 may deliver a first component 204a and a second component 204b for delivery to a patient without combining either component 204a, 204b with a separate component 204c delivered separately from the multi-chamber device 210 (e.g., in a vial 240). Furthermore, it will be appreciated that reference is made to combining the first component 204a with the third component 204c, but the separate component 204c may instead be combined with the second component 204b. It will be appreciated that various principles of the present disclosure may be applied to other combinations and variations of devices, chambers, components, etc.

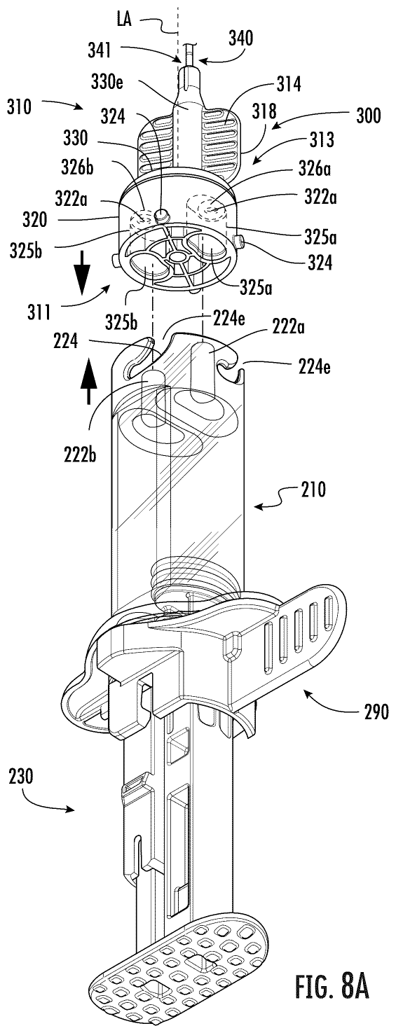

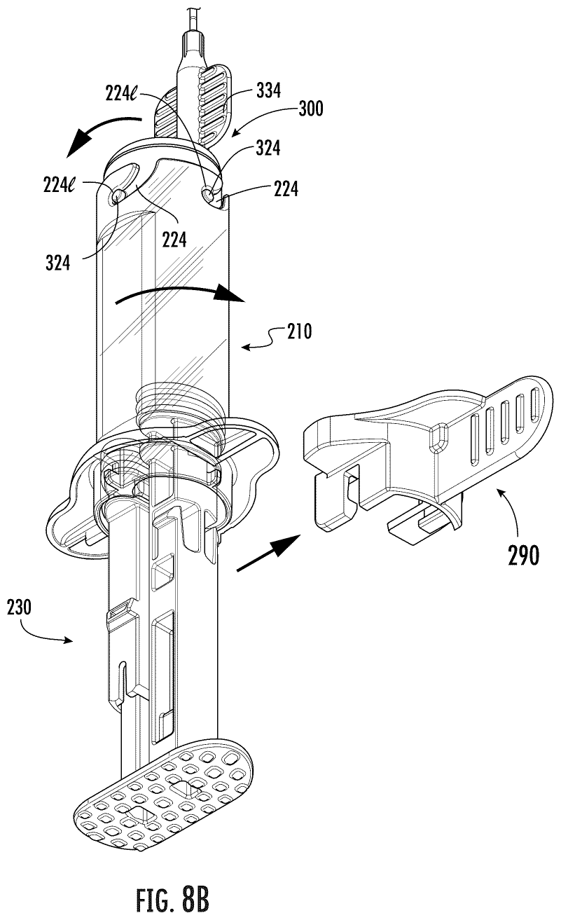

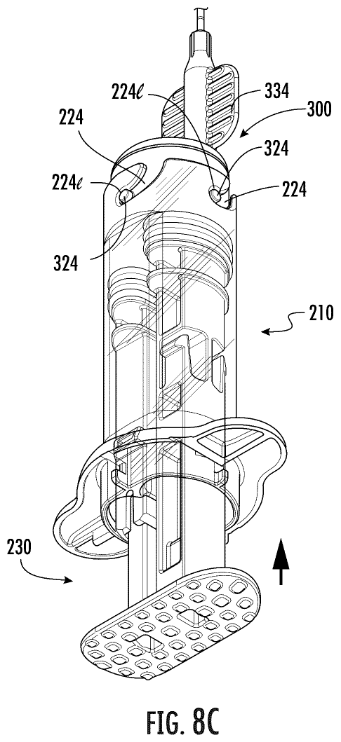

As noted above, a combining and/or delivery system 100 formed in accordance with various principles of the present disclosure includes a device containing material to be delivered to a patient and a separate device configured to deliver the material to the patient. More particularly, in the example of an embodiment illustrated in FIG. 1, a multi-chamber device 210 delivers an injectable material (and optionally combines components thereof) for delivery to a patient via a separate injection system 300. For instance, the multi-chamber system 200 of a combining and/or delivery system 100 formed in accordance with various principles of the present disclosure may not be configured to deliver materials directly to a patient. Instead, the multi-chamber system 200 is configured to be fluidly couplable with an injection system 300 which is configured to deliver the material directly to a patient. In accordance with various principles of the present disclosure, the above-described features of the multi-chamber device 210 facilitating coupling of the multi-chamber device 210 with a vial adaptor 250 may also facilitate coupling of the multi-chamber device 210 with the injection system 300, as will now be described with reference to FIG. 8A, FIG. 8B, and FIG. 8C.

In the example of an embodiment illustrated in FIG. 8A, the injection system 300 includes a base 310 configured to be fluidly coupled with the multi-chamber device 210 so that injectable material from within the multi-chamber device 210 may be delivered to a patient via the injection system 300. More particularly, the base 310 has a proximal end 311 defining an injection system connector portion 320, and a distal end 313 defining an injection system support portion 330 fluidly coupled with the injection system connector portion 320. Furthermore, the injection system support portion 330 includes a support extension 330c configured to support a proximal end 341 of a material delivery device 340. The material delivery device 340 is configured to deliver to a patient the contents of the chambers 202a, 202b of the multi-chamber device 210, injected out through the nozzles 222a, 222b of the multi-chamber device 210 and through the injection system base 310 to the material delivery device 340. More particularly, as may be appreciated with reference to FIG. 9, illustrating an exploded perspective view of the injection system 300 illustrated in FIG. 8A (but at a more proximal angle than as illustrated in FIG. 8A), the injection system connector portion 320 defines injection system nozzle ports 322a, 322b therethrough. The nozzle ports 322a, 322b are configured to be fluidly coupled with the nozzles 222a, 222b of the multi-chamber device 210 as well as with a delivery port 332 defined within the injection system support portion 330. The delivery port 332 is further fluidly coupled with the material delivery device 340. Further details of optional configurations of the delivery port 332 are described below. The material delivery device 340 may have a lumen defined therethrough in fluid communication with the delivery port 332 and through which an injectable material may be delivered to a patient. In the examples of embodiments described herein, the material delivery device 340 may be in the form of a needle which may end at a sharpened distal tip (such as illustrated in FIG. 1). However, it will be appreciated that the present disclosure need not be limited in this regard and other forms and configurations of injection systems and/or material delivery devices are within the scope and spirit of the present disclosure. The above-described fluid communications allow injectable material delivered in the multi-chamber device 210 to be ejected out from the chambers 202a, 202b via the nozzles 222a, 222b thereof, and through the nozzle ports 322a, 322b and the delivery port 332, to the material delivery device 340 which delivers the injectable material to a patient.

Similar to the vial adaptor 250, the injection system 300 optionally includes nozzle pockets 325a, 325b configured to guide the nozzles 222a, 222b of the barrel 220 axially and/or rotationally with respect to the nozzle ports 322a, 322b of the injection system 300 to be in alignment and/or be seated with respect thereto for fluid coupling therewith. The injection system 300 illustrated in FIG. 8A is configured to facilitate fluid coupling and/or to be fluidly coupled with a multi-chamber device 210 in a manner similar to the fluid coupling of the vial adaptor 250 and the multi-chamber device 210 described above. More particularly, in the example of an embodiment illustrated in FIG. 8A, to facilitate interchanging of coupling of the multi-chamber device 210 with the vial adaptor 250 as well as with the injection system 300, the injection system 300 may include barrel-engaging elements 324 similar or substantially identical to those of the vial adaptor 250. For instance, the barrel-engaging elements 324 may be radially-outwardly extending elements which are sized, shaped, configured, and/or dimensioned to engage with the slot-like engaging elements 224 of the multi-chamber device 210. The engaging elements 224 on the multi-chamber device 210 may thus engage and interact with the barrel-engaging elements 324 of the injection system 300 as well with the barrel-engaging elements 254 of the vial adaptor 250 in substantially the same manner, as described in further detail below. As may be appreciated with reference to FIG. 8A, FIG. 8B, and FIG. 8C, one or more (e.g., 1-4, or more) barrel-engaging elements 324 are provided circumferentially around the injection system connector portion 320 of the injection system 300 and are positioned and configured to engage with the one or more engaging elements 224 of the multi-chamber device 210.

As illustrated in FIG. 8A, the multi-chamber device 210 is generally axially aligned with the injection system 300 (e.g., along longitudinal axis LA) so that axial movement of the multi-chamber device 210 and the injection system 300 towards each other allows the barrel-engaging elements 324 of the injection system 300 to become engaged with the engaging elements 224 of the multi-chamber device 210. If necessary, the injection system 300 and the multi-chamber device 210 may be rotated relative to each other to align the barrel-engaging elements 324 of the injection system 300 with the entry opening 224c of the engaging elements 224 of the multi-chamber device 210. The multi-chamber device 210 and the injection system 300 may then be moved closer together (in a generally axially direction) for the barrel-engaging elements 324 of the injection system 300 to enter the engaging elements 224 of the multi-chamber device 210. The multi-chamber device 210 and the injection system 300 may then be rotated with respect to each other (moved rotationally with respect to each other), while also being brought closer together axially, to complete engagement of the barrel-engaging elements 324 of the injection system 300 with the engaging elements 224 of the multi-chamber device 210. In the example of an embodiment illustrated in FIG. 8A-FIG. 8C, the injection system base 310 may include user-engaging elements 314 (such as wings or tabs) facilitating user engagement and manual rotation of the injection system 300 with respect to the multi-chamber device 210. Optionally, the user-engaging elements 314 are provided with surface features 318 (such as bumps, dimples, ribs, grooves, etc.), enhancing grasping thereof. The multi-chamber device 210 and the injection system 300 are thereby brought into the relative positions illustrated in FIG. 8B.

Such configuration of engaging elements 224, 324 as described above provides a relatively simple engagement and alignment of the multi-chamber device 210 and the injection system 300 (similar to the engagement of the vial adaptor 250 and the multi-chamber device 210) into a position as illustrated in FIG. 8B for delivery of the contents of the multi-chamber device 210, via the injection system 300, to a patient. As described above, with respect to the engagement of the multi-chamber device 210 and the vial adaptor 250, the locking section 2240 of the engaging elements 224 of the multi-chamber device 210 may hold the barrel-engaging elements 324 of the injection system 300 in place to resist decoupling of the multi-chamber device 210 and the injection system 300. In some embodiments, such as described above with reference to the vial adaptor 250, a biasing element may be provided to bias the multi-chamber device 210 and the vial adaptor 250 apart from each other to cause the barrel-engaging elements 324 of the injection system 300 to extend into the generally axially-extending locking section 2240 to inhibit unintended relative rotation of the multi-chamber device 210 and the vial adaptor 250. For instance, as described above with respect to the vial adaptor 250 and the multi-chamber device 210, seals 326a, 326b (e.g., O-rings) may be seated with respect to the nozzle ports 322a, 322b of the injection system 300, such as may be appreciated with reference to FIG. 9. Such seals 326a, 326b may bias apart the injection system 300 and the multi-chamber device 210 (when the nozzles 222a, 222b are seated with respect to the injection system nozzle ports 322a, 322b) thereby to maintain the barrel-engaging elements 324 of the injection system 300 seated in the locking section 2240 of the engaging elements 224 of the multi-chamber device 210. As may be appreciated, such seals 326a, 326b may also provide sealing and/or friction such as described above with reference to the seals 256a, 256b of the vial adaptor 250.

As noted above, like the vial adaptor 250, and in accordance with various principles of the present disclosure, the injection system 300 may include nozzle pockets 225a, 225b sized, shaped, configured, and/or dimensioned to guide the nozzles 222a, 222b of the multi-chamber device 210 axially and/or rotationally into alignment with, to be seated with respect to, the nozzle ports 322a, 322b, respectively, of the injection system 300, such as to be in alignment and/or be seated with respect thereto. Such seating may assure that material from the multi-chamber device 210 is directed through the nozzle ports 322a, 322b of the injection system 300 without leakage. An example of an embodiment of a configuration of injection system nozzle pockets 325a, 325b may be appreciated with reference to FIG. 9.

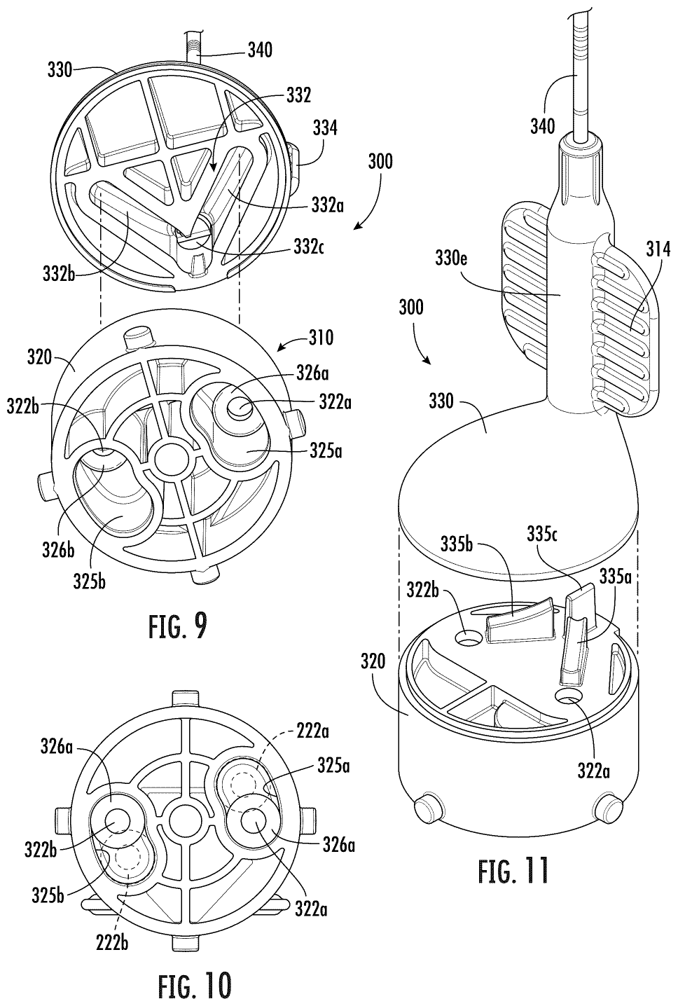

The multi-chamber device 210 and the injection system 300 are illustrated in FIG. 8A and FIG. 8B to be fluidly coupled together with the assistance of the engaging elements 224, 324 and the injection system nozzle pockets 325a, 325b. When the injection system 300 and the multi-chamber device 210 are in the relative positions illustrated in FIG. 8A, the barrel-engaging elements 324 of the injection system 300 are generally aligned with the entry openings 224e of the engaging elements 224 of the multi-chamber device 210 (to facilitate engagement of the engagement elements 224, 324 with one another). However, the proximal end view of the injection system 300 in FIG. 10 illustrates, superimposed in phantom, the positions of the nozzles 222a, 222b with respect to the nozzle ports 325a, 325b when the multi-chamber device 210 and the injection system 300 are oriented with respect to each other as in FIG. 8A. As may be appreciated, the nozzles 222a, 222b are not aligned with the nozzle ports 325a, 325b. The multi-chamber device 210 and the injection system 300 must be rotated with respect to each other along the circumferentially-extending directional arrows illustrated in FIG. 8B for the nozzles 222a, 222b to be aligned and seated with respect to the nozzle ports 325a, 325b, respectively, as well as with the seals 326a, 326b (if provided aligned or seated with respect to the nozzle ports 322a, 322b).

However, in such relative positions of the injection system 300 and the multi-chamber device 210, the nozzles 222a, 222b of the multi-chamber device 210 are not aligned with the nozzle ports 322a, 322b of the injection system 300. As schematically illustrated in the proximal cnd view of the injection system 300 in FIG. 8Bi, the positions of the nozzles 222a, 222b with respect to the nozzle ports 322a, 322b of the injection system 300 and when the multi-chamber device 210 and the injection system 300 are oriented with respect to each other as in FIG. 8B. As may be appreciated with reference to FIG. 8Bi, the nozzles 222a, 222b are not aligned with the nozzle ports 322a, 322b. The multi-chamber device 210 and the injection system 300 must be rotated with respect to each other along the directional arrows illustrated in FIG. 8B for the nozzles 222a, 222b to be aligned and seated with respect to the nozzle ports 322a, 322b, respectively, as well as with the seals 326a, 326b, if provided aligned with the nozzle ports 322a, 322b.

Like the nozzle pockets 255a, 255b of the vial adaptor 250, the nozzle pockets 325a, 325b of the injection system 300 are sized, shaped, configured, and/or dimensioned to facilitate engagement and proper seating of the nozzles 222a, 222b of the multi-chamber device 210 with respective nozzle ports 322a, 322b of the injection system 300. More particularly, the injection system nozzle pockets 325a, 325b are sized, shaped, configured, and/or dimensioned to guide the nozzles 222a, 222b as the barrel-engaging elements 324 of the injection system 300 move along corresponding generally axially-extending segments 224a of the engaging elements 224 of the multi-chamber device 210 (drawing the injection system 300 axially closer to the multi-chamber device 210), and along the generally circumferentially-extending segments 224c of the engaging elements 224 (rotating the nozzles 222a, 222b towards axial alignment with the nozzle ports 322a, 322b of the injection system 300), in a manner similar to the movements described above with reference to fluid coupling of the vial adaptor 250 and the multi-chamber device 210. In view of the similarities, those of ordinary skill in the art may readily understand the relative movements of the various components of the injection system 300 with respect to the multi-chamber device 210 with reference to the above descriptions of the relative movements of the various components of the vial adaptor 250 with respect to the multi-chamber device 210. Furthermore, it will be appreciated that the progression of the nozzles 222a, 222b of the multi-chamber device 210 toward the nozzle ports 322a, 322b of the injection system 300 (as the multi-chamber device 210 and the injection system 300 are moved from the position illustrated in FIG. 8A to the position illustrated in FIG. 8B and into fluid engagement) is similar to the progression of the nozzles 222a, 222b of the multi-chamber device 210 toward the nozzle ports 252a, 252b of the vial adaptor 250 (as depicted in FIG. 5Ai, FIG. 5Aii, FIG. 5Bi, FIG. 5Bii, FIG. 5Ci, and FIG. 5Cii). Accordingly, those of ordinary skill in the art may readily appreciate the relative movements of various elements of the multi-chamber device 210 and the injection system 300 with respect to one another to fluidly couple (or decouple) the multi-chamber device 210 and the injection system 300 with reference to the descriptions of similar relative movements of various elements of the multi-chamber device 210 and the vial adaptor 250 to fluidly couple or decouple such devices. As such, for the sake of brevity, and without intent to limit, reference is made to the above description of the manner in which the engaging elements 254 of the vial adaptor 250 and the engaging elements 224 of the multi-chamber device 210 engage/disengage one another, as well as to above description of the manner in which the vial adaptor nozzle pockets 255a, 255b guide the nozzles 222a, 222b of the multi-chamber device 210 with respect to the nozzle ports 252a, 252b of the vial adaptor 250 as being applicable, mutatis mutandis, to the manner in which the engaging elements 324 of the injection system 300 engage/disengage the engaging elements 224 of the multi-chamber device 210, as well as to the manner in which the injection system nozzle pockets 325a, 325b guide the nozzles 222a, 222b of the multi-chamber device 210 with respect to the nozzle ports 322a, 322b of the injection system 300. It will be appreciated that elements of the injection system 300 corresponding in function to elements of the vial adaptor 250 are indicated with similar reference numerals increased by 100.

Once the multi-chamber device 210 is securely coupled with the injection system 300, and the nozzles 222a, 222b of the multi-chamber device 210 are seated with respect to the nozzle ports 322a, 322b of the injection system 300, the retainer 290 may be withdrawn from the plunger assembly 230, such as illustrated in FIG. 8B. The plunger assembly 230 may then be advanced distally, toward the nozzles 222a, 222b at the distal end 213 of the multi-chamber device 210, as illustrated in FIG. 8C, to eject the precursor 204d and the second component 204b therefrom and into a patient via the injection system 300.

As described above, in embodiments of a combining and/or delivery system 100 with a multi-chamber device 210 (in contrast with a single chamber device), it may be desirable for the components 204a, 204b within the chambers 202a, 202b of the multi-chamber device 210 to remain separate until the time of delivery to a patient. In some embodiments, the components 204a, 204b react with each other, and, therefore, it is desirable for such components 204a, 204b to remain separate for as long as possible. In some embodiments, such components 204a, 204b are combined just prior to (e.g., immediately prior to) delivery to the patient so that the reaction begins, or at least is completed, within the patient. In some embodiments, initial combining of the components 204a, 204b delivered by the multi-chamber device 210 may occur within the injection system support portion 330, or within the material delivery device 340, or within the patient.

In accordance with various principles of the present disclosure, to facilitate fluid coupling of the multi-chamber device nozzles 222a, 222b and the injection system nozzle ports 322a, 322b with the material delivery device 340 (which typically has only a single fluid passage, such as a single lumen), the delivery port 332 defined in the injection system support portion 330 may be configured as/with a Y-connector, as may be seen with reference to the perspective view in FIG. 9. For instance, the injection system nozzle ports 322a, 322b defined in the illustrated example of an embodiment of an injection system connector portion 320 are respectively fluidly coupled with Y-connector branches 332a, 332b defined in the illustrated example of an embodiment of an injection system support portion 330. The branches 332a, 332b of the delivery port 332 meet and flow into a common lumen 332c defined in the injection system support portion 330 and in fluid communication with a lumen defined through the material delivery device 340 for delivering the injectable material into a patient in a manner such as known those of ordinary skill in the art. It is noted that in the example of an embodiment illustrated in FIG. 8A and FIG. 9, the multi-chamber device nozzles 222a, 222b, the injection system nozzle ports 322a, 322b, and the branches 332a, 332b of the delivery port 332 are illustrated generally equidistantly spaced apart from the longitudinal axis LA of the system, and the common lumen 332c is illustrated generally aligned (e.g., coaxial) with the longitudinal axis LA of the system. However, asymmetrical arrangements/configurations are within the scope and spirit of the present disclosure.

In the example of an embodiment of an injection system base 310 illustrated in FIG. 9, the injection system connector portion 320 and the injection system support portion 330 are formed separately (such as illustrated), such as to facilitate manufacture thereof. However, it will be appreciated that the present disclosure need not be limited in this regard (e.g., the injection system base 310 may be formed as a single, monolithic element defining a connector portion 320 at a proximal end 311 thereof and a support portion 330 at a distal end 313 thereof). Nonetheless, if the injection system connector portion 320 and the injection system support portion 330 are formed separately, as in the example of an embodiment illustrated in FIG. 9, then the injection system connector portion 320 may include one or more alignment elements 335a, 335b, 335c configured for engagement with the delivery port 332 defined in the injection system support portion 330, such as in the example of an embodiment illustrated in FIG. 11. More particularly, the example of embodiments of alignment elements 335a, 335b illustrated in FIG. 11 are configured to project into the branches 332a, 332b of the delivery port 332 defined in the injection system support portion 330. Additionally or alternatively, the example of an embodiment of an alignment element 335c illustrated in FIG. 11 is configured to project into the common lumen 332c of the delivery port 332 defined in the injection system support portion 330. The alignment elements 335a, 335b, 335c may be positioned, sized, shaped, configured, and/or dimensioned to align and/or to stabilize the injection system connector portion 320 and the injection system support portion 330 with respect to each other (such as during or after assembly thereof), such as to maintain fluid communication therebetween without leakage. In some aspects, elements 335a, 335b may reduce dead volume within the injection system 300, such as by taking up space in the fluid pathway, so more injectable material is delivered to the patient instead of remaining in the combining and/or delivery system 100. The alignment elements 335a, 335b, 335c are positioned, sized, shaped, configured, and/or dimensioned with respect to the branches 332a, 332b and/or common lumen 332c of the delivery port 332 so as not to interfere with fluid flow from the nozzle ports 322a, 322b in the injection system connector portion 320 and the delivery port 332 in the injection system support portion 330, as may be appreciated by those of ordinary skill in the art.

In view of the above, it will be appreciated that the multi-chamber device 210 may be configured not just to facilitate coupling the multi-chamber device 210 with an optional vial adaptor 250 and additional chamber 204c formed in a separate device 240, but also to facilitate couple of the multi-chamber device 210 with the injection system 300 of the combining and/or delivery system 100 illustrated in FIG. 1. Similarly, the injection system 300 may be configured not just to be coupled with the multi-chamber device 210, but may also be configured to be coupled with one or more other injectable material transfer devices, such as one or more other material-containing devices. For instance, as described above, in some instances, it may be desirable to administer a pre-treatment material to a patient before delivering an injectable material from the multi-chamber device 210 to the patient. In accordance with various principles of the present disclosure, the engaging elements 324 of the injection system base 310 may be configured not only to engage the engaging elements 224 of the multi-chamber device 210, but also to engage with engaging elements facilitating coupling of the injection system 300 with other material-containing devices, such as other devices with chambers containing injectable materials. Optionally, the other material-containing devices have engaging elements with configurations similar to the engaging elements 224 of the multi-chamber device 210 to facilitate coupling with the injection system 300.

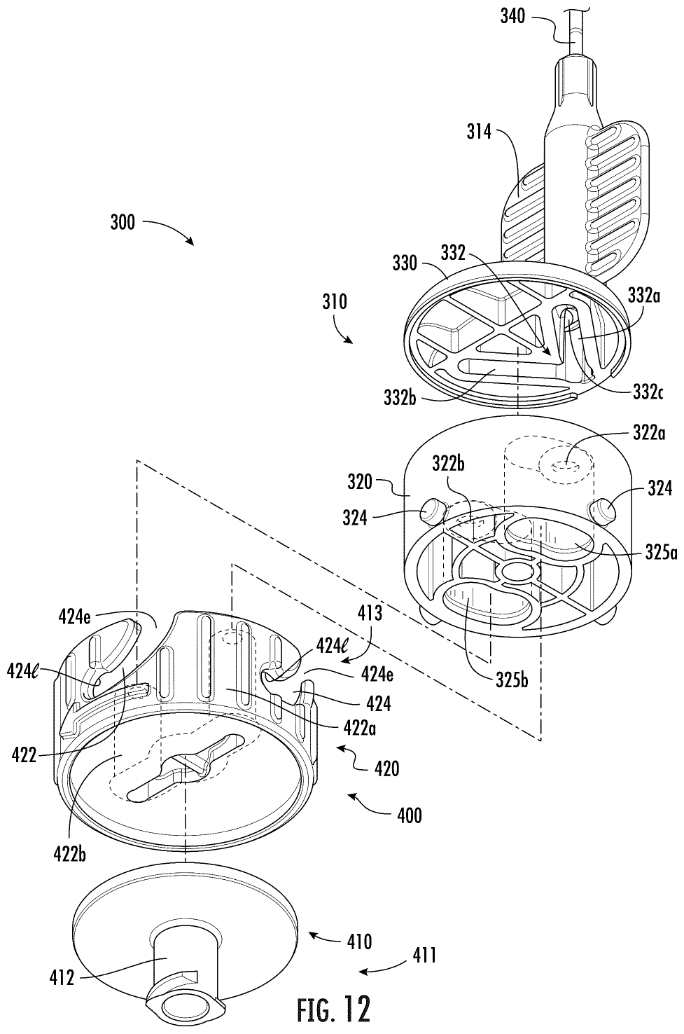

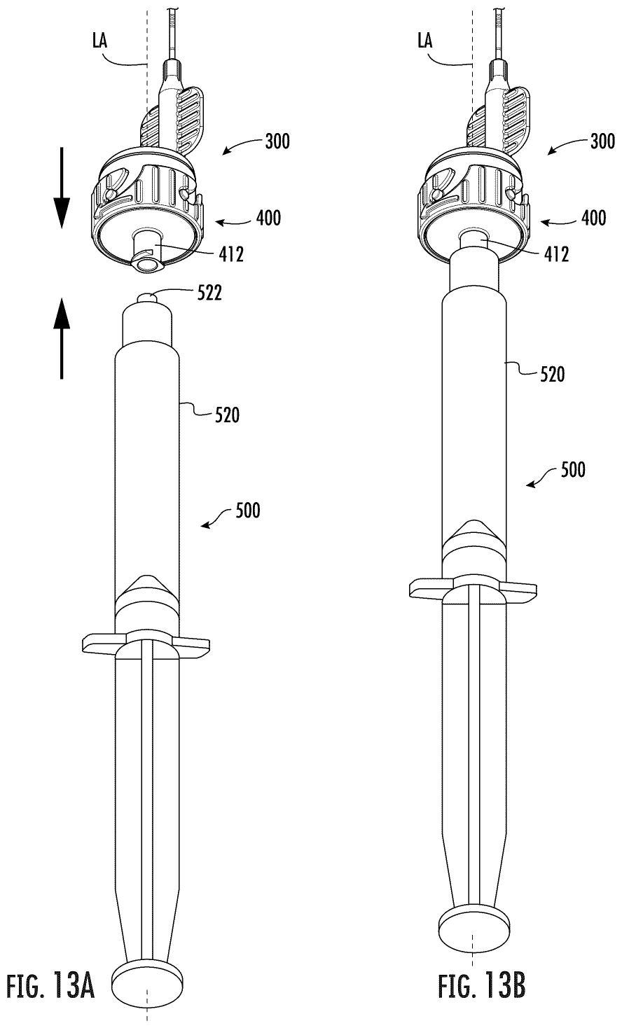

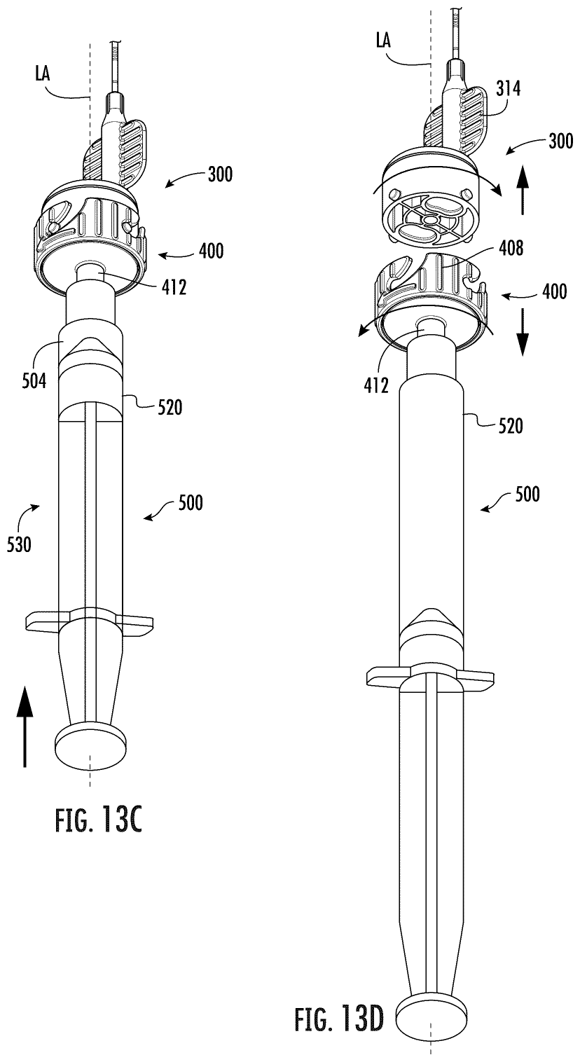

In some aspects, an injection system 300 formed in accordance with various principles of the present disclosure is configured to be fluidly coupled with a material-containing device having a single fluid outlet (referenced herein as a nozzle for the sake of convenience and without intent to limit) configured to eject an injectable material into patient via the injection system 300. In such instances, a combining and/or delivery system 100 formed in accordance with various principles of the present disclosure may include an adaptor system 400, configured to adapt an injection system 300 with two ports 322a, 322b to be able to fluidly communicate with a material transfer device 500 with a single nozzle, such as illustrated in FIG. 1, FIG. 12, and FIGS. 13A-13D. In some instances, such device is a pretreatment device delivering a pretreatment material to a patient via the injection system 300. However, the present disclosure need not be limited in this regard.

An example of an embodiment of an adaptor system 400 formed in accordance with various principles of the present disclosure is illustrated in FIG. 12 as having an inlet portion 410 and an outlet portion 420. The illustrated example of an embodiment of an inlet portion 410 has a single port 412 extending proximally therefrom toward the proximal end 411 of the adaptor system 400. The illustrated example of an embodiment of an outlet portion 420 has a pair of nozzles 422a, 422b extending distally therefrom toward the distal end 413 of the adaptor system 400. It will be appreciated that the inlet portion 410 and the outlet portion 420 of an adaptor-system 400 formed in accordance with various principles of the present disclosure may be formed separately (such as illustrated), or as a single, monolithic element, the present disclosure not being limited in this regard. The single port 412 of the adaptor system 400 is configured to be fluidly coupled with a material transfer device 500 having a barrel 520 with a single nozzle 522, such as illustrated in FIGS. 13A-13D. The pair of nozzles 422a, 422b of the adaptor system 400 are configured to be fluidly coupled, respectively, with the injection system nozzle ports 322a, 322b, as may be appreciated with reference to FIG. 12.

In accordance with various principles of the present disclosure, the injection system 300 of a combining and/or delivery system 100 formed in accordance with various principles of the present disclosure may utilize the same engaging elements 324 for engaging both the multi-chamber device 210 as well as the adaptor system 400 (and, via the adaptor system 400, the material transfer device 500), such as to facilitate case of use, to simplify the combining and/or delivery system 100, etc. For instance, in the example of an embodiment illustrated in FIG. 12, the adaptor system 400 has one or more (e.g., 1-4, or more) engaging elements 424 similar or substantially identical to the engaging elements 224 of the multi-chamber device 210. More particularly, the engaging elements 424 of the adaptor-system outlet portion 420 may be configured as slots similar to the engaging elements 224 of the multi-chamber device 210. The adaptor system 400 may thus be coupled with the injection system 300 by axial and/or rotational movements similar to those described above with respect to the injection system 300 and the multi-chamber device 210. Moreover, the injection system nozzle pockets 325a, 325b may guide the nozzles 422a, 422b of the adaptor system 400 axially and/or rotationally with respect to the injection system nozzle ports 322a, 322b in substantially the same manner as described above with respect to the injection system nozzle pockets 325a, 325b guiding the multi-chamber device nozzles 222a, 222b into alignment to seat with the nozzle ports 252a, 252b. Accordingly, for the sake of brevity, and without intent to limit, reference is made to the above description of the manner in which the engaging elements 324 of the injection system 300 and the engaging elements 224 of the multi-chamber device 210 engage/disengage one another, as well as to above description of the manner in which the injection system nozzle pockets 325a, 325b guide the nozzles 222a, 222b of the multi-chamber device 210 as being applicable, mutatis mutandis, to the manner in which the engaging elements 324 of the injection system 300 engage/disengage the adaptor system engaging elements 424, as well as to the manner in which the injection system nozzle pockets 325a, 325b guide the nozzles 422a, 422b of the adaptor system 400. It will be appreciated that elements of the adaptor system 400 corresponding in function to elements of the multi-chamber device 210 are indicated with similar reference numerals increased by 200.

Once the adaptor system 400 has been coupled with the injection system 300, the nozzle 522 of the material transfer device 500 may be aligned with the port 412 of the adaptor system 400, as illustrated in FIG. 13A. The material-containing device nozzle 522 may then be moved into engagement with the adaptor-system port 412, along the axially-extending directional arrows illustrated in FIG. 13A, to fluidly couple the material transfer device 500 with the injection system 300 with the assistance of the adaptor system 400, such as illustrated in FIG. 13B. The plunger 530 of the material transfer device 500 may then be advanced with respect to the material transfer device 500. For instance, the plunger 530 of the material transfer device 500 may be advanced distally into the chamber 502 of the material transfer device 500, as illustrated in FIG. 13C, to expel material 504 out from the chamber 502 for delivery to a patient via the adaptor system 400 and the injection system 300.

The adaptor system 400 may be separated from the injection system 300 (e.g., after delivery of material 504 out from the material transfer device 500) by reversing the movements performed to couple the adaptor system 400 and the injection system 300. For instance, the injection system 300 and the adaptor system 400 may be rotated with respect to each other (moved rotationally with respect to each other along the circumferentially-extending directional arrows illustrated in FIG. 13D) to move the engaging elements 324, 424 to a position in which the injection system 300 and the adaptor system 400 may be moved generally axially apart (along the axially-extending directional arrows illustrated in FIG. 13D) to be separated/disengaged from each other. It will be appreciated that the user-engaging elements 314 of the injection system base 310 may facilitate user engagement and manual rotation of the injection system 300 with respect to the adaptor system 400. The adaptor system 400 may be provided with surface features 408 (such as ribs, grooves, bumps, dimples, etc.) enhancing grasping thereof, in a manner known to those of ordinary skill in the art. For the sake of brevity, and without intent to limit, reference is made to the above description of the manner in which the engaging elements 324 of the injection system 300 and the engaging elements 224 of the multi-chamber device 210 are disengaged with respect to one another as being applicable, mutatis mutandis, to the manner in which the engaging elements 324 of the injection system 300 are disengaged with respect to the adaptor system engaging elements 424. Optionally, as illustrated in FIG. 13D, the material transfer device 500 may remain coupled with the adaptor system 400 to be separated/disengaged from the injection system 300 together with the adaptor system 400 (e.g., separated from the injection system 300 at the same time the adaptor system 400 is separated from the injection system 300). However, it will be appreciated that the material transfer device 500 may, instead, be separated/disengaged from the adaptor system 400 before the adaptor system 400 is separated/disengaged from the injection system 300.

In view of the above, it will be appreciated that, in contrast with prior systems having multiple components which need to be delivered, assembled with respect to one another, separated from one another, manipulated together with and separately from one another, etc., the present disclosure simplifies devices, systems, and methods for combining and/or delivering an injectable material to a patient and/or reduces (if not eliminates) human error occurring with the use of complex prior systems.

Further in view of the above, it should be understood that the various embodiments illustrated in the figures have several separate and independent features, which each, at least alone, has unique benefits which are desirable for, yet not critical to, the presently disclosed devices, systems, and methods. Therefore, the various separate features described herein need not all be present in order to achieve at least some of the desired characteristics and/or benefits described herein. For instance, only one of the various features described above may be present in a device or system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An injectable material delivery system configured to establish fluid communication between first and second injectable material transfer devices thereof, said system comprising:
a first injectable material transfer device having a first nozzle; and
a second injectable material transfer device having a first nozzle port configured to be seated with respect to said first nozzle of said first injectable material transfer device, and a first nozzle pocket configured to guide said first nozzle of said first injectable material transfer device as said first injectable material transfer device and said second injectable material transfer device are brought together generally axially and are rotated with respect to each other to seat said first nozzle of said first injectable material transfer device with respect to said first nozzle port of said second injectable material transfer.

2. The system of claim 1, wherein said first injectable material transfer device and said second injectable material transfer device have engaging elements configured to couple said first injectable material transfer device and said second injectable material transfer device by relative axial and/or rotational movements of said first injectable material transfer device and said second injectable material transfer device, with the guidance of said first nozzle pocket.

3. The system of claim 2, further comprising a biasing element positioned between said first injectable material transfer device and said second injectable material transfer device to bias said first injectable material transfer device and said second injectable material transfer device with respect to each other to lock said engaging elements of said first injectable material transfer device and said second injectable material transfer device with respect to each other.

4. The system of claim 2, further comprising a third injectable material transfer device having engaging elements configured to be similar to engaging elements of said second injectable material transfer device to engage engaging elements of said first injectable material transfer device.

5. The system of claim 4, wherein said third injectable material transfer device has a first nozzle port configured to be seated with respect to said first nozzle of said first injectable material transfer device, and a first nozzle pocket configured to guide said first nozzle of said first injectable material transfer device as said first injectable material transfer device and said third injectable material transfer device are brought together generally axially and are rotated with respect to each other to seat said first nozzle of said first injectable material transfer device with respect to said first nozzle port of said third injectable material transfer device.

6. The system of claim 5, further comprising a fourth injectable material transfer device having engaging elements configured to be similar to engaging elements of said first injectable material transfer device to engage engaging elements of said second injectable material transfer device.

7. The system of claim 4, further comprising a fourth injectable material transfer device having engaging elements configured to be similar to engaging elements of said first injectable material transfer device to engage engaging elements of said second injectable material transfer device.

8. The system of claim 1, wherein said first injectable material transfer device is selected from the group consisting of: a device defining a material chamber therein and having at least one nozzle through which material is ejected therefrom or aspirated therein; a multi-chamber device; a single-chamber device; a device defining a nozzle port with respect to which a nozzle of another device may be seated in fluid communication therewith; a vial adaptor; an injection system; a pretreatment system; or an adaptor device or system coupling or facilitating coupling of a first device with at least one nozzle with a second device with at least one nozzle port.

9. The system of claim 1, wherein:
said first injectable material transfer device is a multi-chamber device defining a first chamber and a second chamber therein;
said first injectable material transfer device has a second nozzle;
said first nozzle of said first injectable material transfer device is in fluid communication with the first chamber of said first injectable material transfer device; and
said second nozzle of said first injectable material transfer device is in fluid communication with the second chamber of said first injectable material transfer device.

10. The system of claim 9, wherein:
said first nozzle port of said second injectable material transfer device is configured to be seated with respect to said first nozzle of said first injectable material transfer device;
said second injectable material transfer device has a second nozzle port configured to be seated with respect to said second nozzle of said first injectable material transfer device; and
said second injectable material transfer device has a second nozzle pocket configured to guide said second nozzle of said first injectable material transfer device to seat with respect to said second nozzle port of said second injectable material transfer device as said first injectable material transfer device and said second injectable material transfer device are brought together generally axially and are rotated with respect to each other to seat said second nozzle of said first injectable material transfer device with respect to said second nozzle port of said second injectable material transfer.

11. The system of claim 10, wherein said second injectable material transfer device comprises an injection system comprising a material delivery device configured to deliver injectable material ejected from said first injectable material transfer device and through said second injectable material transfer device to a patient.

12. The system of claim 9, wherein said second injectable material transfer device comprises an injection system comprising a material delivery device configured to deliver injectable material ejected from said first injectable material transfer device and through said second injectable material transfer device to a patient.

13. The system of claim 1, wherein said first injectable material transfer device has a barrel defining a first chamber therein, and a plunger assembly movable with respect to the first chamber to eject material from or to aspirate material into the first chamber.

14. The system of claim 13, wherein said second injectable material transfer device comprises an injection system comprising a material delivery device configured to deliver injectable material ejected from said first nozzle of said first injectable material transfer device and through said injection system to a patient.

15. The system of claim 14, further comprising a vial adaptor having:
a vial-receiving chamber configured to receive a vial therein;
a first nozzle port configured to be seated with respect to said first nozzle of said first injectable material transfer device; and
a first nozzle pocket configured to guide said first nozzle of said first injectable material transfer device as said first injectable material transfer device and said vial adaptor are brought together generally axially and are rotated with respect to each other to seat said first nozzle of said first injectable material transfer device with respect to said first nozzle port of said vial adaptor.

16. The system of claim 15, further comprising an adaptor system having a single nozzle port configured to be fluidly coupled with a single-nozzle device, and a first nozzle configured to be guided by said first nozzle pocket of said injection system as said adaptor system and said injection system are brought together generally axially and are rotated with respect to each other to seat said first nozzle of said adaptor system with respect to said first nozzle port of said injection system.

17. The system of claim 14, further comprising an adaptor system having a single nozzle port configured to be fluidly coupled with a single-nozzle device, and a first nozzle configured to be guided by said first nozzle pocket of said injection system as said adaptor system and said injection system are brought together generally axially and are rotated with respect to each other to seat said first nozzle of said adaptor system with respect to said first nozzle port of said injection system.

18. An injectable material transfer device configured for coupling with another injectable material transfer device having a first nozzle, said injectable material transfer device comprising:
a first nozzle port configured to be seated with respect to a first nozzle of the other injectable material transfer device; and
a first nozzle pocket configured to guide the first nozzle of the other injectable material transfer device as said injectable material transfer device and the other injectable material transfer device are brought together generally axially and are rotated with respect to each other to seat the first nozzle of the other injectable material transfer device with respect to said first nozzle port of said injectable material transfer device.

19. The device of claim 18, further comprising engaging elements configured to engage said device with engaging elements of the other injectable material transfer device, wherein said device and the other injectable material transfer device move generally axially as well as rotationally with respect to each other as said engaging elements of said device and the engaging elements of the other injectable material transfer device are moved with respect to each other.

20.

21. A method for coupling a first injectable material transfer device with a second injectable material transfer device, said method comprising:
advancing a nozzle of the first injectable material transfer device axially into a nozzle pocket of the second injectable material transfer device; and
moving the first injectable material device and the second injectable material transfer device rotationally and generally axially within nozzle pockets configured to guide movement of the first nozzle into alignment and to seat with a first nozzle port defined in the second injectable material transfer device.
